(19) United States
(12) Patent Application Publication
(10) Pub. No.: US 2022/0390348 A1
(43) Pub. Date: Dec. 8, 2022

(54) FLOW CELLS AND METHODS

(71) Applicant: ILLUMINA, INC., San Diego, CA (US)

(72) Inventors: Eric M. Brustad, San Diego, CA (US); Craig Michael Ciesla, Mountain View, CA (US); Jeffrey S. Fisher, San Diego, CA (US); Sahngki Hong, San Diego, CA (US); Lewis J. Kraft, San Diego, CA (US)

(21) Appl. No.: 17/826,091

(22) Filed: May 26, 2022

Related U.S. Application Data
(60) Provisional application No. 63/195,123, filed on May 31, 2021.

(51) Int. Cl. G01N 15/14; C12Q 1/6844; C12Q 1/6869

(52) U.S. Cl. CPC G01N 15/1404; C12Q 1/6844; C12Q 1/6869; C12Q 2600/16

(57) ABSTRACT

An example of a flow cell includes a substrate; a plurality of reactive regions extending along the substrate; and a non-reactive region separating one of the plurality of reactive regions from an adjacent one of the plurality of reactive regions. Each of the plurality of reactive regions includes alternating first and second areas positioned along the reactive region. Each of the first areas includes a first primer set and each of the second areas includes a second primer set that is different than the first primer set. Either adjacent first and second areas directly abut each other, or) the first areas are positioned on protrusions and the second areas are positioned in depressions adjacent to the protrusions.

### CROSS REFERENCE TO RELATED APPLICATION

This application claims the benefit of U.S. Provisional Application Ser. No. 63/195,123, filed May 31, 2021, the contents of which is incorporated by reference herein in its entirety.

### REFERENCE TO SEQUENCE LISTING

The Sequence Listing submitted via EFS-Web is hereby incorporated by reference in its entirety. The name of the file is ILI213B_IP-2091-US_Sequence_Listing_ST25.txt, the size of the file is 3,001 bytes, and the date of creation of the file is May 26, 2022.

### BACKGROUND

Some available platforms for sequencing nucleic acids utilize a sequencing-by-synthesis approach. With this approach, a nascent strand is synthesized, and the addition of each monomer (e.g., nucleotide) to the growing strand is detected optically and/or electronically. Because a template strand directs synthesis of the nascent strand, one can infer the sequence of the template DNA from the series of nucleotide monomers that were added to the growing strand during the synthesis. In some examples, sequential paired-end sequencing may be used, where forward strands are sequenced and removed, and then reverse strands are constructed and sequenced.

### SUMMARY

Some of the examples disclosed herein are flow cells that include different primer sets at adjacent reactive areas. The different primer sets are orthogonal to each other. Orthogonal primer sets enable different template strands to be amplified on the respective reactive areas, without enabling a template strand from an adjacent reactive area to seed and amplify. As such, orthogonal primers reduce or eliminate index or pad hopping, which is the contamination of a cluster of amplicons of one library template in one reactive area with a different amplicon from another reactive area. Orthogonal primers also enable the reactive areas to be arranged in close proximity to one another, with little to no interstitial region separating the reactive areas. This increases the reactive area density, and thus the cluster density; and also decreases non-functional space on the flow cell surface. Increased reactive area density increases the signal intensity during sequencing.

### BRIEF DESCRIPTION OF THE DRAWINGS

Features of examples of the present disclosure will become apparent by reference to the following detailed description and drawings, in which like reference numerals correspond to similar, though perhaps not identical, components. For the sake of brevity, reference numerals or features having a previously described function may or may not be described in connection with other drawings in which they appear.

FIG. 1A is a schematic view of example active regions including different primer sets;

FIG. 1B through FIG. 1E are schematic views of primer sets that enable the generation of forward and reverse strands on adjacent active regions;

FIG. 2 is a top view of a flow cell;

FIG. 3A and FIG. 3C are semi-schematic, perspective views of different examples of the surface chemistry architecture in the flow cell, each of which includes reactive regions separated by non-reactive regions;

FIG. 3B is a cross-sectional view, taken along line 3B-3B of FIG. 3A, of one of the reactive reactions;

FIG. 4A and FIG. 4C are semi-schematic, perspective views of different examples of the surface chemistry architecture in the flow cell, each of which includes rows and columns of alternating first and second reactive areas;

FIG. 4B is a cross-sectional view, taken along line 4B-4B of FIG. 4A, of one of the rows of alternating first and second reactive areas;

FIG. 5 is a semi-schematic, perspective view of another example of the surface chemistry architecture in the flow cell, which includes alternating regions of a first height and a second height, and alternating first and second reactive areas extending along the regions;

FIG. 6 is a semi-schematic, perspective view of another example of the surface chemistry architecture in the flow cell, which includes three different reactive areas;

FIG. 7A is a top view of yet another example of the surface chemistry architecture in the flow cell, which includes capture primers at designated locations;

FIG. 7B, FIG. 7C, and FIG. 7D are cross-sectional views of different examples the architecture in FIG. 7A;

FIG. 8A through FIG. 8C together schematically depict an example of a method for making some examples of the surface chemistry architecture disclosed herein;

FIG. 9A through FIG. 9D together schematically depict an example of another method for making some examples of the surface chemistry architecture disclosed herein;

FIG. 10A through FIG. 10C together schematically depict an example of yet another method for making other examples of the surface chemistry architecture disclosed herein;

FIG. 11A through FIG. 11D together schematically depict an example of still another method for making some examples of the surface chemistry architecture disclosed herein;

FIG. 12A through FIG. 12L together schematically depict an example of still another method for making some examples of the surface chemistry architecture disclosed herein;

FIG. 13A is a top view of the surface chemistry architecture formed by the method of FIG. 12A through FIG. 12L; and

FIG. 13B is a schematic perspective view of a portion of a multi-depth substrate used in the method of FIG. 12A through FIG. 12L, illustrating the hexagonal shaped geometry of the surfaces where the different surface chemistries are introduced.

### DETAILED DESCRIPTION

Some of the flow cells disclosed herein include orthogonal primer sets at adjacent reactive areas. Orthogonal primer sets enable different template strands to be amplified on the respective reactive areas, without enabling a template strand from an adjacent reactive area to seed and amplify. As such, orthogonal primers reduce or eliminate index or pad hopping. Orthogonal primers also increase the reactive area density, and thus signal intensity during sequencing.

Other flow cells disclosed herein include orthogonal capture primers arranged in rows and offset columns across the substrate. Orthogonal capture primers enable different template strands to be seeded at respective areas across the substrate surface, and a surrounding primer set enables the seeded template strands to be amplified.

### Definitions

It is to be understood that terms used herein will take on their ordinary meaning in the relevant art unless specified otherwise. Several terms used herein and their meanings are set forth below.

The singular forms “a”, “an”, and “the” include plural referents unless the context clearly dictates otherwise.

The terms comprising, including, containing and various forms of these terms are synonymous with each other and are meant to be equally broad.

The terms top, bottom, lower, upper, on, etc. are used herein to describe the flow cell and/or the various components of the flow cell. It is to be understood that these directional terms are not meant to imply a specific orientation, but are used to designate relative orientation between components. The use of directional terms should not be interpreted to limit the examples disclosed herein to any specific orientation(s).

The terms first, second, etc. also are not meant to imply a specific orientation or order, but rather are used to distinguish one component from another.

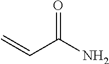

An “acrylamide monomer” is a monomer with the structure or a monomer including an acrylamide group. Examples of the monomer including an acrylamide group include azido acetamido pentyl acrylamide:

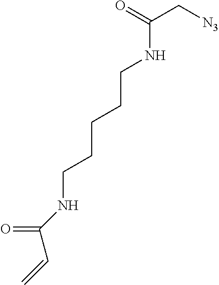 [SMILES: C=CC(=O)NCCCCCNC(=O)CN=[N+]=[N-]]

### and N-isopropylacrylamide:

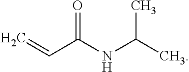

Other acrylamide monomers may be used.

The term “activation,” as used herein, refers to a process that generates reactive groups at the surface of a base support or an outermost layer of a multi-layered structure. Activation may be accomplished using silanization or plasma ashing. While activation may be performed in each of the methods disclosed herein, the figures do not depict the reactive groups. It is to be understood, however, that a silanized layer or —OH groups (from plasma ashing) are present to covalently attach the polymeric hydrogels to the underlying support or layer. Suitable silanes that may be used in a silanization process include amino silanes, such as (3-aminopropyl)trimethoxysilane) (APTMS), (3-aminopropyl)triethoxysilane) (APTES), N-(6-aminohexyl)aminomethyltriethoxysilane (AHAMTES), N-(2-aminoethyl)-3-aminopropyltriethoxysilane (AEAPTES), and N-(2-aminoethyl)-3-aminopropyltrimethoxysilane (AEAPTMS); alkynyl silanes, such as O-propargyl)-N-(triethoxysilylpropyl)carbamate, cyclooctyne, a cyclooctyne derivative, or bicyclononynes (e.g., bicyclo[6.1.0]non-4-yne or derivatives thereof, bicyclo[6.1.0]non-2-yne, or bicyclo[6.1.0]non-3-yne); or norbornene silanes, such as [(5-bicyclo[2.2.1]hept-2-enyl)ethyl]trimethoxysilane.

An aldehyde, as used herein, is an organic compound containing a functional group with the structure —CHO, which includes a carbonyl center (i.e., a carbon double-bonded to oxygen) with the carbon atom also bonded to hydrogen and an R group, such as an alkyl or other side chain. The general structure of an aldehyde is:

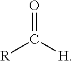

As used herein, “alkyl” refers to a straight or branched hydrocarbon chain that is fully saturated (i.e., contains no double or triple bonds). The alkyl group may have 1 to 20 carbon atoms. Example alkyl groups include methyl, ethyl, propyl, isopropyl, butyl, isobutyl, tertiary butyl, pentyl, hexyl, and the like. As an example, the designation “C1-4 alkyl” indicates that there are one to four carbon atoms in the alkyl chain, i.e., the alkyl chain is selected from the group consisting of methyl, ethyl, propyl, iso-propyl, n-butyl, isobutyl, sec-butyl, and t-butyl.

As used herein, “alkenyl” refers to a straight or branched hydrocarbon chain containing one or more double bonds. The alkenyl group may have 2 to 20 carbon atoms. Example alkenyl groups include ethenyl, propenyl, butenyl, pentenyl, hexenyl, and the like.

As used herein, “alkyne” or “alkynyl” refers to a straight or branched hydrocarbon chain containing one or more triple bonds. The alkynyl group may have 2 to 20 carbon atoms.

As used herein, “aryl” refers to an aromatic ring or ring system (i.e., two or more fused rings that share two adjacent carbon atoms) containing only carbon in the ring backbone. When the aryl is a ring system, every ring in the system is aromatic. The aryl group may have 6 to 18 carbon atoms. Examples of aryl groups include phenyl, naphthyl, azulenyl, and anthracenyl.

An “amino” functional group refers to an —NRₐR_{b} group, where Rₐ and R_{b} are each independently selected from hydrogen (e.g.,

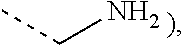

C1-6 alkyl, C2-6 alkenyl, C2-6 alkynyl, C3-7 carbocycle, C6-10 aryl, 5-10 membered heteroaryl, and 5-10 membered heterocycle, as defined herein.

As used herein, the term “attached” refers to the state of two things being joined, fastened, adhered, connected or bound to each other, either directly or indirectly. For example, a nucleic acid can be attached to a polymeric hydrogel by a covalent or non-covalent bond. A covalent bond is characterized by the sharing of pairs of electrons between atoms. A non-covalent bond is a physical bond that does not involve the sharing of pairs of electrons and can include, for example, hydrogen bonds, ionic bonds, van der Waals forces, hydrophilic interactions and hydrophobic interactions.

An “azide” or “azido” functional group refers to —N₃.

As used herein, a “bonding region” refers to an area of a patterned structure that is to be bonded to another material, which may be, as examples, a spacer layer, a lid, another patterned structure, etc., or combinations thereof (e.g., a spacer layer and a lid, or a spacer layer and another patterned structure). The bond that is formed at the bonding region may be a chemical bond (as described above), or a mechanical bond (e.g., using a fastener, etc.).

As used herein, “carbocycle” means a non-aromatic cyclic ring or ring system containing only carbon atoms in the ring system backbone. When the carbocycle is a ring system, two or more rings may be joined together in a fused, bridged or spiro-connected fashion. Carbocycles may have any degree of saturation, provided that at least one ring in a ring system is not aromatic. Thus, carbocycles include cycloalkyls, cycloalkenyls, and cycloalkynyls. The carbocycle group may have 3 to 20 carbon atoms. Examples of carbocycle rings include cyclopropyl, cyclobutyl, cyclopentyl, cyclohexyl, cyclohexenyl, 2,3-dihydro-indene, bicyclo[2.2.2]octanyl, adamantyl, and spiro[4.4]nonanyl.

As used herein, the term “carboxylic acid” or “carboxyl” as used herein refers to —COOH.

As used herein, “cycloalkylene” means a fully saturated carbocycle ring or ring system that is attached to the rest of the molecule via two points of attachment.

As used herein, “cycloalkenyl” or “cycloalkene” means a carbocycle ring or ring system having at least one double bond, wherein no ring in the ring system is aromatic. Examples include cyclohexenyl or cyclohexene and norbornenyl or norbornene. Also as used herein, “heterocycloalkenyl” or “heterocycloalkene” means a carbocycle ring or ring system with at least one heteroatom in ring backbone, having at least one double bond, wherein no ring in the ring system is aromatic.

As used herein, “cycloalkynyl” or “cycloalkyne” means a carbocycle ring or ring system having at least one triple bond, wherein no ring in the ring system is aromatic. An example is cyclooctyne. Another example is bicyclononyne. Also as used herein, “heterocycloalkynyl” or “heterocycloalkyne” means a carbocycle ring or ring system with at least one heteroatom in ring backbone, having at least one triple bond, wherein no ring in the ring system is aromatic.

The term “depositing,” as used herein, refers to any suitable application technique, which may be manual or automated, and, in some instances, results in modification of the surface properties. Generally, depositing may be performed using vapor deposition techniques, coating techniques, grafting techniques, or the like. Some specific examples include chemical vapor deposition (CVD), spray coating (e.g., ultrasonic spray coating), spin coating, dunk or dip coating, doctor blade coating, puddle dispensing, flow through coating, aerosol printing, screen printing, microcontact printing, inkjet printing, or the like.

As used herein, the term “depression” refers to a discrete concave feature in a base support or a layer of a multi-layer stack. Depressions can have any of a variety of shapes at their opening in a surface including, as examples, round, elliptical, square, polygonal, star shaped (with any number of vertices), etc. The cross-section of a depression taken orthogonally with the surface can be curved, square, polygonal, hyperbolic, conical, angular, etc.

The term “each,” when used in reference to a collection of items, is intended to identify an individual item in the collection, but does not necessarily refer to every item in the collection. Exceptions can occur if explicit disclosure or context clearly dictates otherwise.

The term “epoxy” (also referred to as a glycidyl or oxirane group) as used herein refers to

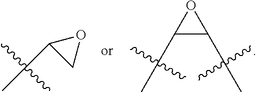

As used herein, the term “flow cell” is intended to mean a vessel having a flow channel where a reaction can be carried out, an inlet for delivering reagent(s) to the flow channel, and an outlet for removing reagent(s) from the flow channel. In some examples, the flow cell accommodates the detection of the reaction that occurs in the flow cell. For example, the flow cell can include one or more transparent surfaces allowing for the optical detection of arrays, optically labeled molecules, or the like.

As used herein, a “flow channel” or “channel” may be an area defined between two bonded components, which can selectively receive a liquid sample. In some examples, the flow channel may be defined between two patterned structures, and thus may be in fluid communication with the surface chemistry of each of the patterned structures. In other examples, the flow channel may be defined between a patterned structure and a lid, and thus may be in fluid communication with the surface chemistry of the one patterned structure.

As used herein, “heteroaryl” refers to an aromatic ring or ring system (i.e., two or more fused rings that share two adjacent atoms) that contain(s) one or more heteroatoms, that is, an element other than carbon, including but not limited to, nitrogen, oxygen and sulfur, in the ring backbone. When the heteroaryl is a ring system, every ring in the system is aromatic. The heteroaryl group may have 5-18 ring members.

As used herein, “heterocycle” means a non-aromatic cyclic ring or ring system containing at least one heteroatom in the ring backbone. Heterocycles may be joined together in a fused, bridged or spiro-connected fashion. Heterocycles may have any degree of saturation provided that at least one ring in the ring system is not aromatic. In the ring system, the heteroatom(s) may be present in either a non-aromatic or aromatic ring. The heterocycle group may have 3 to 20 ring members (i.e., the number of atoms making up the ring backbone, including carbon atoms and heteroatoms). In some examples, the heteroatom(s) are O, N, or S.

The term “hydrazine” or “hydrazinyl” as used herein refers to a —NHNH₂ group.

As used herein, the term “hydrazone” or “hydrazonyl” as used herein refers to a

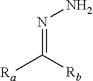

group in which Rₐ and R_{b} are each independently selected from hydrogen, C1-6 alkyl, C2-6 alkenyl, C2-6 alkynyl, C3-7 carbocycle, C6-10 aryl, 5-10 membered heteroaryl, and 5-10 membered heterocycle, as defined herein.

As used herein, “hydroxy” or “hydroxyl” refers to an —OH group.

As used herein, the term “interstitial region” refers to an area, e.g., of a base support or a layer of a multi-layer stack that separates depressions (concave regions) or protrusions (convex regions). In the examples disclosed herein, some of the depressions and/or protrusions directly abut one another and thus there is no interstitial region present between the two abutting depressions and/or protrusions. In other examples, an interstitial region may be formed between depressions and/or protrusions that are positioned at a diagonal to one other, or may be formed at the intersection of three of four abutting depressions and/or protrusions. Interstitial regions have a surface material that differs from the surface material of the depressions or the protrusions. For example, the depressions and/or protrusions can have a polymeric hydrogel and a primer set thereon, while the interstitial region is free of this surface chemistry.

As used herein, a “negative photoresist” refers to a light-sensitive material in which a portion that is exposed to light of particular wavelength(s) becomes insoluble to a developer. In these examples, the insoluble negative photoresist has less than 5% solubility in the developer. With the negative photoresist, the light exposure changes the chemical structure so that the exposed portions of the material becomes less soluble (than non-exposed portions) in the developer. While not soluble in the developer, the insoluble negative photoresist may be at least 99% soluble in a remover that is different from the developer. The remover may be a solvent or solvent mixture used, e.g., in a lift-off process.

In contrast to the insoluble negative photoresist, any portion of the negative photoresist that is not exposed to light is at least 95% soluble in the developer. In some examples, the portion of the negative photoresist not exposed to light is at least 98%, e.g., 99%, 99.5%, 100%, soluble in the developer.

“Nitrile oxide,” as used herein, means a “RₐC≡N⁺O⁻” group in which Rₐ is defined herein. Examples of preparing nitrile oxide include in situ generation from aldoximes by treatment with chloramide-T or through action of base on imidoyl chlorides [RC(Cl)═NOH] or from the reaction between hydroxylamine and an aldehyde.

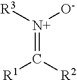

“Nitrone,” as used herein, means a group in which R¹, R², and R³ may be any of the Rₐ and R_{b} groups defined herein, except that R³ is not hydrogen (H).

As used herein, a “nucleotide” includes a nitrogen containing heterocyclic base, a sugar, and one or more phosphate groups. Nucleotides are monomeric units of a nucleic acid sequence. In RNA, the sugar is a ribose, and in DNA, the sugar is a deoxyribose, i.e. a sugar lacking a hydroxyl group that is present at the 2′ position in ribose. The nitrogen containing heterocyclic base (i.e., nucleobase) can be a purine base or a pyrimidine base. Purine bases include adenine (A) and guanine (G), and modified derivatives or analogs thereof. Pyrimidine bases include cytosine (C), thymine (T), and uracil (U), and modified derivatives or analogs thereof. The C-1 atom of deoxyribose is bonded to N-1 of a pyrimidine or N-9 of a purine. A nucleic acid analog may have any of the phosphate backbone, the sugar, or the nucleobase altered. Examples of nucleic acid analogs include, for example, universal bases or phosphate-sugar backbone analogs, such as peptide nucleic acid (PNA).

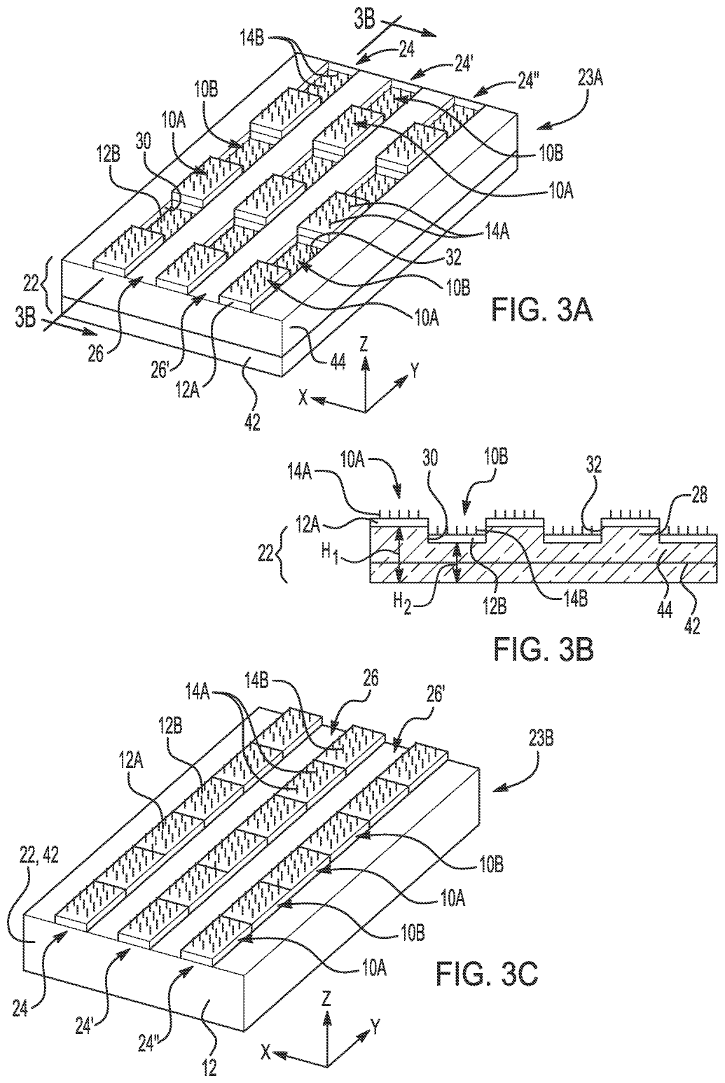

In some examples, the term “over” may mean that one component or material is positioned directly on another component or material. When one is directly on another, the two are in contact with each other. In FIG. 3A, the layer 44 is applied over the base support 42 so that it is directly on and in contact with the base support 42.

In other examples, the term “over” may mean that one component or material is positioned indirectly on another component or material. By indirectly on, it is meant that a gap or an additional component or material may be positioned between the two components or materials. In FIG. 3A, the polymeric hydrogel 12A is positioned over the base support 42 such that the two are in indirect contact. More specifically, the polymeric hydrogel 12A is indirectly on the base support 42 because the resin layer 44 is positioned between the two components 12A and 42.

A “patterned resin” refers to any polymer that can have depressions and/or protrusions defined therein. Specific examples of resins and techniques for patterning the resins will be described further below.

A “patterned structure” refers to a single layer base support that includes, or a multi-layer stack with a layer that includes surface chemistry in a pattern, e.g., in depressions, on protrusions, or otherwise positioned on the support or layer surface. The surface chemistry may include a polymeric hydrogel and primers. In some examples, the single layer base support or the layer of the multi-layer stack have been exposed to patterning techniques (e.g., etching, lithography, etc.) in order to generate the pattern for the surface chemistry. However, the term “patterned structure” is not intended to imply that such patterning techniques have to be used to generate the pattern. For example, a base support may be a substantially flat surface having a pattern of the polymeric hydrogel thereon. The patterned structure may be generated via any of the methods disclosed herein.

As used herein, the “primer” is defined as a single stranded nucleic acid sequence (e.g., single strand DNA). Some primers, referred to herein as capture primers, serve as a seed for a library template. Some other primers, referred to herein as amplification primers, serve as a starting point for template amplification and cluster generation. In some instances, an amplification primer may also serve as a capture primer for seeding a library template. Still other primers, referred to herein as sequencing primers, serve as a starting point for DNA synthesis. The 5′ terminus of the primer may be modified to allow a coupling reaction with a functional group of a polymer. The primer length can be any number of bases long and can include a variety of non-natural nucleotides. In an example, the sequencing primer is a short strand, ranging from 10 to 60 bases, or from 20 to 40 bases.

As used herein, a “positive photoresist” refers to a light-sensitive material in which a portion that is exposed to light of particular wavelength(s) becomes soluble to a developer. In these examples, any portion of the positive photoresist exposed to light is at least 95% soluble in the developer. In some examples, the portion of the positive photoresist exposed to light is at least 98%, e.g., 99%, 99.5%, 100%, soluble in the developer. With the positive photoresist, the light exposure changes the chemical structure so that the exposed portions of the material become more soluble (than non-exposed portions) in the developer.

In contrast to the soluble positive photoresist, any portion of the positive photoresist not exposed to light is insoluble (less than 5% soluble) in the developer. While not soluble in the developer, the insoluble positive photoresist may be at least 99% soluble in a remover that is different from the developer. In some examples, insoluble positive photoresist is at least 98%, e.g., 99%, 99.5%, 100%, soluble in the remover. The remover may be a solvent or solvent mixture used in a lift-off process.

A “spacer layer,” as used herein refers to a material that bonds two components together. In some examples, the spacer layer can be a radiation-absorbing material that aids in bonding, or can be put into contact with a radiation-absorbing material that aids in bonding.

The term “substrate” refers to the single layer base support or a multi-layer structure upon which surface chemistry is introduced.

The term “surface chemistry” refers to a polymeric hydrogel and a primer set attached to the polymeric hydrogel. The surface chemistry may be arranged in a variety of architectures in the examples disclosed herein. The surface chemistry makes up a reactive area or region of the substrate, and areas or regions of the substrate that are free of the surface chemistry may be referred to as non-reactive areas or regions.

The term “tantalum pentoxide” refers to the inorganic compound with the formula Ta₂O₅. This compound is transparent, having a transmittance ranging from about 0.25 (25%) to 1 (100%), to wavelengths ranging from about 0.35 μm (350 nm) to at least 1.8 μm (1800 nm). A “tantalum pentoxide base support” or “tantalum pentoxide layer” may comprise, consist essentially of, or consist of Ta₂O₅.

A “thiol” functional group refers to —SH.

As used herein, the terms “tetrazine” and “tetrazinyl” refer to six-membered heteroaryl group comprising four nitrogen atoms. Tetrazine can be optionally substituted.

“Tetrazole,” as used herein, refer to five-membered heterocyclic group including four nitrogen atoms. Tetrazole can be optionally substituted.

Primers

In some of the examples set forth herein, the flow cell includes different primer sets attached to immediately adjacent reactive areas. In one example, immediately adjacent reactive areas are those that are next to each other on a substrate surface and that abut one another. In another example, immediately adjacent reactive areas are those that are next to each on a substrate surface, but that do not abut one another because they are positioned at different heights.

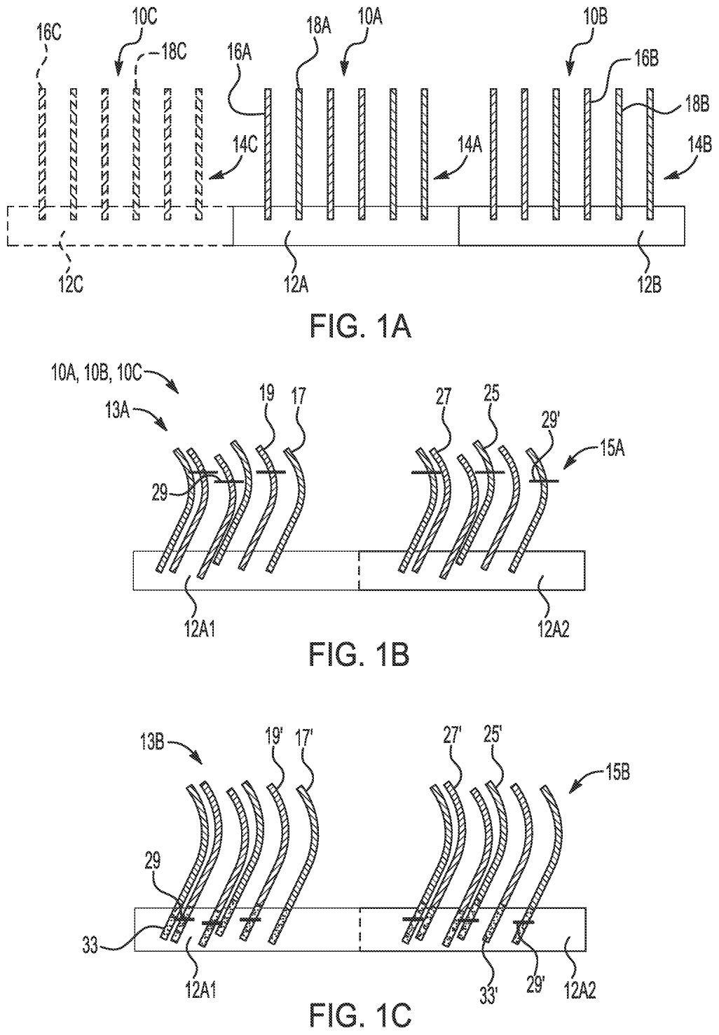

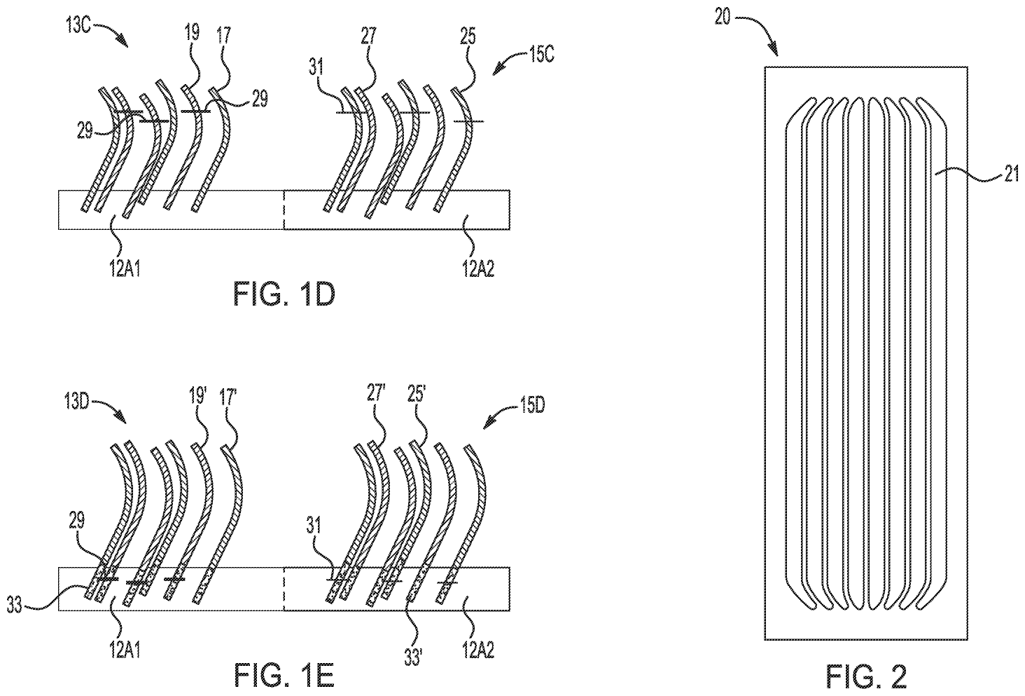

Examples of different primer sets attached to immediately adjacent reactive areas are shown in FIG. 1. Each of the reactive areas 10A, 10B, 10C includes a polymeric hydrogel 12A, 12B, 12C to which the respective primer set 14A, 14B, 14C is attached. As will be described in more detail below, the polymeric hydrogel 12A, 12B, 12C in one reactive area 10A, 10B, 10C may be the same as or different than the polymeric hydrogel 12A, 12B, 12C in another reactive area 10A, 10B, 10C.

Each primer set 14A, 14B, 14C includes two different primers 16A, 18A, or 16B, 18B, or 16C, 18C, such as forward and reverse amplification primers. The primers 16A, 18A of the first set, e.g., 14A, together enable the amplification of a library template having end adapters that are complementary to the two different primers 16A, 18A in that first set 14A. The primers 16B, 18B of a second set, e.g., 16B, together enable the amplification of a different library template having end adapters that are complementary to the two different primers 16B, 18B in the second set 14B, but do not enable the library template associated with the first primer set 14A to be seeded or amplified. In some examples, a third primer set 14C is used. In these examples, the primers 16C, 18C of the third set 14C together enable the amplification of a different library template having end adapters that are complementary to the two different primers 16C, 18C in the third set 14C, but do not enable the library template associated with the first primer set 14A or the second primer set 14B to be seeded or amplified.

As examples, the first primer set 12 includes P5 and P7 primers; and the second primer set 14 includes any combination of the PA primers, the PB primers, the PC primers, and the PD primers set forth herein. In other examples, P15 and P7 may be used in the first primer set 12. As examples, the second primer set 14 may include any two (or three) PA, PB, PC, and PD primers, or any combination of one PA primer and one PB, PC, or PD primer, or any combination of one PB primer and one PC or PD primer, or any combination of one PC primer and one PD primer.

Examples of P5 and P7 primers are used on the surface of commercial flow cells sold by Illumina Inc. for sequencing, for example, on HISEQ™, HISEQX™, MISEQ™, MISEQDX™, MINISEQ™, NEXTSEQ™, NEXTSEQDX™, NOVASEQ™, ISEQ™, GENOME ANALYZER™, and other instrument platforms. The P5 primer is:

P5: 5′→3′
(SEQ. ID. NO. 1)
AATGATACGGCGACCACCGAGAUCTACAC
The P7 primer may be any of the following:
P7 #1: 5′→3′
(SEQ. ID. NO. 2)
CAAGCAGAAGACGGCATACGAnAT
P7 #2: 5′→3′
(SEQ. ID. NO. 3)
CAAGCAGAAGACGGCATACnAGAT

where “n” is 8-oxoguanine or uracil in each of these sequences.
The P15 primer is:

P15: 5′→3′
(SEQ. ID. NO. 4)
AATGATACGGCGACCACCGAGAnCTACAC

where “n” is allyl-T.
The other primers (PA-PD) mentioned above include:

PA 5′→3′
(SEQ. ID. NO. 5)
GCTGGCACGTCCGAACGCTTCGTTAATCCGTTGAG
cPA (PA′) 5′→3′
(SEQ. ID. NO. 6)
CTCAACGGATTAACGAAGCGTTCGGACGTGCCAGC
PB 5′→3′
(SEQ. ID. NO. 7)
CGTCGTCTGCCATGGCGCTTCGGTGGATATGAACT
cPB (PB′) 5′→3′
(SEQ. ID. NO. 8)
AGTTCATATCCACCGAAGCGCCATGGCAGACGACG
PC 5′→3′
(SEQ. ID. NO. 9)
ACGGCCGCTAATATCAACGCGTCGAATCCGCAACT
cPC (PC′) 5′→3′
(SEQ. ID. NO. 10)
AGTTGCGGATTCGACGCGTTGATATTAGCGGCCGT
PD 5′→3′
(SEQ. ID. NO. 11)
GCCGCGTTACGTTAGCCGGACTATTCGATGCAGC
cPD (PD′) 5′→3′
(SEQ. ID. NO. 12)
GCTGCATCGAATAGTCCGGCTAACGTAACGCGGC

While not shown in the example sequences for PA-PD, it is to be understood that any of these primers may include a cleavage site, such as uracil, 8-oxoguanine, allyl-T, etc. at any point in the strand.

As other examples, the first primer set 14A includes unblocked P5 and P7 primers (SEQ. ID. NO. 1, SEQ. ID. NO. 2 or SEQ. ID. NO. 3); and the second primer set 14B includes 3′ blocked P5 and P7 primers. In this example, a blocking group (e.g., a 3′ phosphate) may be added that attaches to the exposed 3′ ends of the primers 16B, 18B of the second primer set 14B. The blocking group prevents undesired extension at these primers 16B, 18B during the amplification of the first primer set 14A. The blocking group may then be removed to perform a round of amplification with a newly added library template and the second primer set 14B.

In still other examples, one primer set 14A, 14B, 14C described herein may be used with another primer set which enables simultaneous paired end sequencing. Primer sets that enable simultaneous paired end sequencing include primer subsets on different regions of the polymeric hydrogel. FIG. 1B through FIG. 1E depict different configurations of the primer subsets 13A, 15A, 13B, 15B, 13C, 15C, and 13D, 15D attached to the polymeric hydrogel regions 12A1, 12A2.

Each of the first primer subsets 13A, 13B, 13C, and 13D includes an un-cleavable first primer 17 or 17′ and a cleavable second primer 19 or 19′; and each of the second primer subsets 15A, 15B, 15C, and 15D includes a cleavable first primer 25 or 25′ and an un-cleavable second primer 27 or 27′.

The un-cleavable first primer 17 or 17′ and the cleavable second primer 19 or 19′ are oligonucleotide pairs, e.g., where the un-cleavable first primer 17 or 17′ is a forward amplification primer and the cleavable second primer 19 or 19′ is a reverse amplification primer or where the cleavable second primer 19 or 19′ is the forward amplification primer and the un-cleavable first primer 17 or 17′ is the reverse amplification primer. In each example of the first primer subset 13A, 13B, 13C, and 13D, the cleavable second primer 19 or 19′ includes a cleavage site 29, while the un-cleavable first primer 17 or 17′ does not include a cleavage site 29.

The cleavable first primer 25 or 25′ and the un-cleavable second primer 27 or 27′ are also oligonucleotide pairs, e.g., where the cleavable first primer 25 or 25′ is a forward amplification primer and the un-cleavable second primer 27 or 27′ is a reverse amplification primer or where the un-cleavable second primer 27 or 27′ is the forward amplification primer and the cleavable first primer 25 or 25′ is the reverse amplification primer. In each example of the second primer subset 15A, 15B, 15C, and 15D, the cleavable first primer 25 or 25′ includes a cleavage site 29′ or 31, while the un-cleavable second primer 27 or 27′ does not include a cleavage site 29′ or 31.

It is to be understood that the un-cleavable first primer 17 or 17′ of the first primer subset 13A, 13B, 13C, and 13D and the cleavable first primer 25 or 25′ of the second primer subset 15A, 15B, 15C, and 15D have the same nucleotide sequence (e.g., both are forward amplification primers), except that the cleavable first primer 25 or 25′ includes the cleavage site 29′ or 31 integrated into the nucleotide sequence or into a linker 33′ attached to the nucleotide sequence. Similarly, the cleavable second primer 19 or 19′ of the first primer subset 13A, 13B, 13C, and 13D and the un-cleavable second primer 27 or 27′ of the second primer subset 15A, 15B, 15C, and 15D have the same nucleotide sequence (e.g., both are reverse amplification primers), except that the cleavable second primer 19 or 19′ includes the cleavage site 29 integrated into the nucleotide sequence or into a linker 33 attached to the nucleotide sequence.

It is to be understood that when the first primers 17 and 25 or 17′ and 25′ are forward amplification primers, the second primers 19 and 27 or 19′ and 27′ are reverse primers, and vice versa.

The un-cleavable primers 17, 27 or 17′, 27′ may be any primers with a universal sequence for capture and/or amplification purposes, such as the P5, P7, and P15 primers or any combination of the PA, PD, PC, PD primers (e.g., PA and PB or PA and PD, etc.). It is to be understood that these primers 17, 27 or 17′, 27′ would not include the cleavage site (e.g., uracil, 8-oxoguanine, etc.) shown in the sequences. In some examples, the P5 and P7 primers are un-cleavable primers 17, 27 or 17′, 27′ because they do not include, respectively, the uracil and 8-oxoguanine. It is to be understood that any suitable universal sequence can be used as the un-cleavable primers 17, 27 or 17′, 27′.

Examples of cleavable primers 19, 25 or 19′, 25′ include the P5 and P7 primers or other universal sequence primers (e.g., the PA, PB, PC, PD primers) with the respective cleavage sites 29, 29′, 31 incorporated into the respective nucleic acid sequences (e.g., FIG. 1B and FIG. 1D), or into a linker 33′, 33 that attaches the cleavable primers 19, 25 or 19′, 25′ to the respective functionalized layers 24, 26 or layer pads 24′, 26′ (FIG. 1C and FIG. 1E). Examples of suitable cleavage sites 29, 29′, 31 include enzymatically cleavable nucleobases or chemically cleavable nucleobases, modified nucleobases, or linkers (e.g., between nucleobases), as described herein.

Each primer subset 13A and 15A or 13B and 15B or 13C and 15C or 13D and 15D is attached to a polymeric hydrogel region 12A1, 12A2. The polymeric hydrogel regions 12A1, 12A2 may include different functional groups that can selectively react with the respective primers 17, 19 or 17′, 19′ or 25, 27 or 25′, 27′.

While not shown in FIG. 1B through FIG. 1E, it is to be understood that one or both of the primer subsets 13A, 13B, 13C, or 13D or 15A, 15B, 15C or 15D may also include a PX primer for capturing/seeding a library template. As one example, PX may be included with the primer subsets 13A, 13B, 13C, and 13D, but not with primer subsets 15A, 15B, 15C or 15D. As another example, PX may be included with the primer subsets 13A, 13B, 13C, and 13D and with the primer subsets 15A, 15B, 15C or 15D. The density of the PX motifs should be relatively low in order to minimize polyclonality within each depression 30 (see, e.g., FIG. 3B).

FIG. 1B through FIG. 1E depict different configurations of the primer subsets 13A, 15A, 13B, 15B, 13C, 15C, and 13D, 15D attached to the polymeric hydrogel region 12A1, 12A2. More specifically, FIG. 1B through FIG. 1E depict different configurations of the primers 17, 19 or 17′, 19′ and 25, 27 or 25′, 27′ that may be used.

In the example shown in FIG. 1B, the primers 17, 19 and 25, 27 of the primer subsets 13A and 15A are directly attached to the polymeric hydrogel regions 12A1, 12A2, for example, without a linker 33, 33′. The polymeric hydrogel region 12A1 has surface functional groups that can immobilize the terminal groups at the 5′ end of the primers 17, 19. Similarly, the polymeric hydrogel region 12A2 has surface functional groups that can immobilize the terminal groups at the 5′ end of the primers 25, 27. The immobilization chemistry between the polymeric hydrogel region 12A1 and the primers 17, 19 and the immobilization chemistry between the polymeric hydrogel region 12A2 and the primers 25, 27 is different so that the primers 17, 19 or 25, 27 selectively attach to the desirable polymeric hydrogel region 12A1, 12A2. The polymeric hydrogel regions 12A1, 12A2 may include any example polymeric hydrogel 12A, 12B, 12C disclosed herein.

Also, in the example shown in FIG. 1B, the cleavage site 29, 29′ of each of the cleavable primers 19, 25 is incorporated into the sequence of the primer. In this example, the same type of cleavage site 29, 29′ is used in the cleavable primers 19, 25 of the respective primer subsets 13A, 15A. As an example, the cleavage sites 29, 29′ are uracil bases, and the cleavable primers 19, 25 are P5U (see SEQ. ID. NO. 1) and P7U (see SEQ. ID. NOS. 2 and 3). The uracil bases or other cleavage sites may also be incorporated into any of the PA, PB, PC, and PD primers to generate the cleavable primers 19, 25. In this example, the un-cleavable primer 17 of the oligonucleotide pair 17, 19 may be P7 (without 8-oxoguanine), and the un-cleavable primer 27 of the oligonucleotide pair 25, 27 may be P5 (without uracil). Thus, in this example, the first primer subset 13A includes P7 (without 8-oxoguanine), P5U (SEQ. ID. NO. 1) and the second primer subset 15A includes P5 (without uracil), P7U (SEQ. ID. NO. 2 or NO. 3). The primer subsets 13A, 15A have opposite linearization chemistries, which, after amplification, cluster generation, and linearization, allows forward template strands to be formed on one polymeric hydrogel region 12A1 and reverse strands to be formed on the other polymeric hydrogel region 12A2.

In the example shown in FIG. 1C, the primers 17′, 19′ and 25′, 27′ of the primer subsets 13B and 15B are attached to the polymeric hydrogel regions 12A1, 12A2, for example, through linkers 33, 33′. The polymeric hydrogel regions 12A1, 12A2 include respective functional groups, and the terminal ends of the respective linkers 33, 33′ are capable of covalently attaching to the respective functional groups. As such, the polymeric hydrogel region 12A1 may have surface functional groups that can immobilize the linker 33 at the 5′ end of the primers 17′, 19′. Similarly, the polymeric hydrogel region 12A2 may have surface functional groups that can immobilize the linker 33′ at the 5′ end of the primers 25′, 27′. The immobilization chemistry for the polymeric hydrogel region 12A1 and the linkers 33 and the immobilization chemistry for the polymeric hydrogel region 12A2 and the linkers 33′ is different so that the primers 17′, 19′ or 25′, 27′ selectively graft to the desirable polymeric hydrogel regions 12A1, 12A2.

Examples of suitable linkers 33, 33′ may include nucleic acid linkers (e.g., 10 nucleotides or less) or non-nucleic acid linkers, such as a polyethylene glycol chain, an alkyl group or a carbon chain, an aliphatic linker with vicinal diols, a peptide linker, etc. An example of a nucleic acid linker is a polyT spacer, although other nucleotides can also be used. In one example, the spacer is a 6T to 10T spacer. The following are some examples of nucleotides including non-nucleic acid linkers with terminal alkyne groups (where B is the nucleobase and “oligo” is the primer):

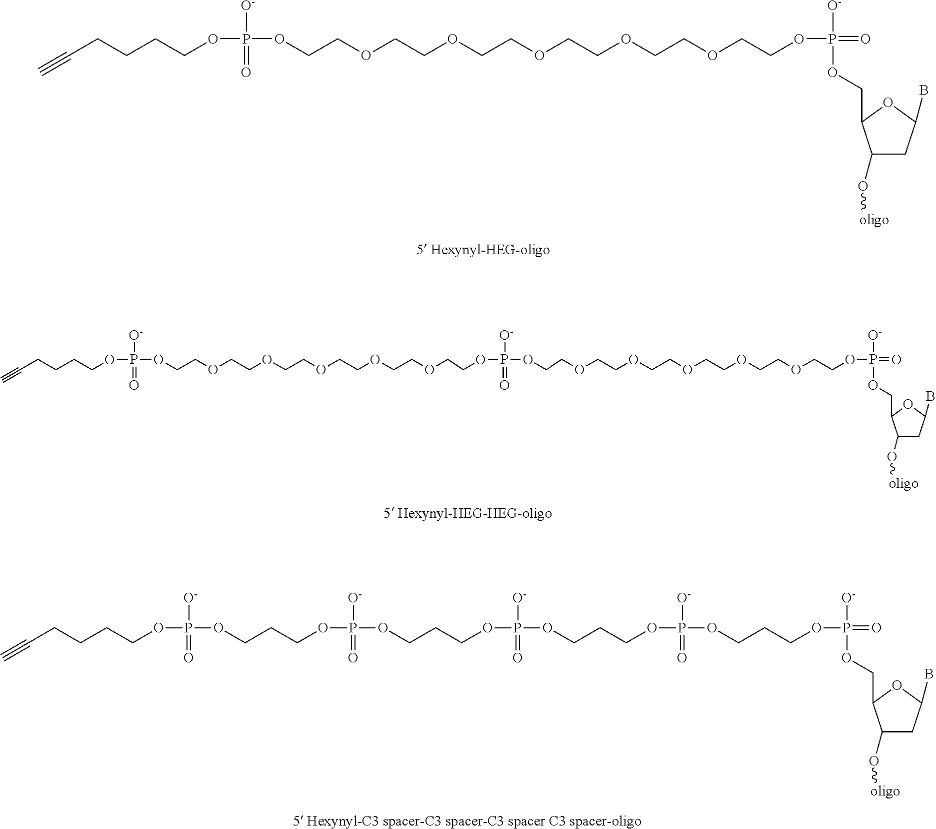

In the example shown in FIG. 1C, the primers 17′, 25′ have the same sequence (e.g., P5 without uracil) and the same or different linker 33, 33′. The primer 17′ is un-cleavable, whereas the primer 25′ includes the cleavage site 29′ incorporated into the linker 33′. Also in this example, the primers 19′, 27′ have the same sequence (e.g., P7 without 8-oxoguanine) and the same or different linker 33, 33′. The primer 27′ in un-cleavable, and the primer 19′ includes the cleavage site 29 incorporated into the linker 33. The same type of cleavage site 29, 29′ is used in the linker 33, 33′ of each of the cleavable primers 19′, 25′. As an example, the cleavage sites 29, 29′ may be uracil bases that are incorporated into nucleic acid linkers 33, 33′. The primer subsets 13B, 15B have opposite linearization chemistries, which, after amplification, cluster generation, and linearization, allows forward template strands to be formed on one polymeric hydrogel region 12A1 and reverse strands to be formed on the other polymeric hydrogel region 12A2.

The example shown in FIG. 1D is similar to the example shown in FIG. 1B, except that different types of cleavage sites 29, 31 are used in the cleavable primers 19, 25 of the respective primer subsets 13C, 15C. As examples, two different enzymatic cleavage sites may be used, two different chemical cleavage sites may be used, or one enzymatic cleavage site and one chemical cleavage site may be used. Examples of different cleavage sites 29, 31 that may be used in the respective cleavable primers 19, 25 include any combination of the following: vicinal diol, uracil, allyl ether, disulfide, restriction enzyme site, and 8-oxoguanine.

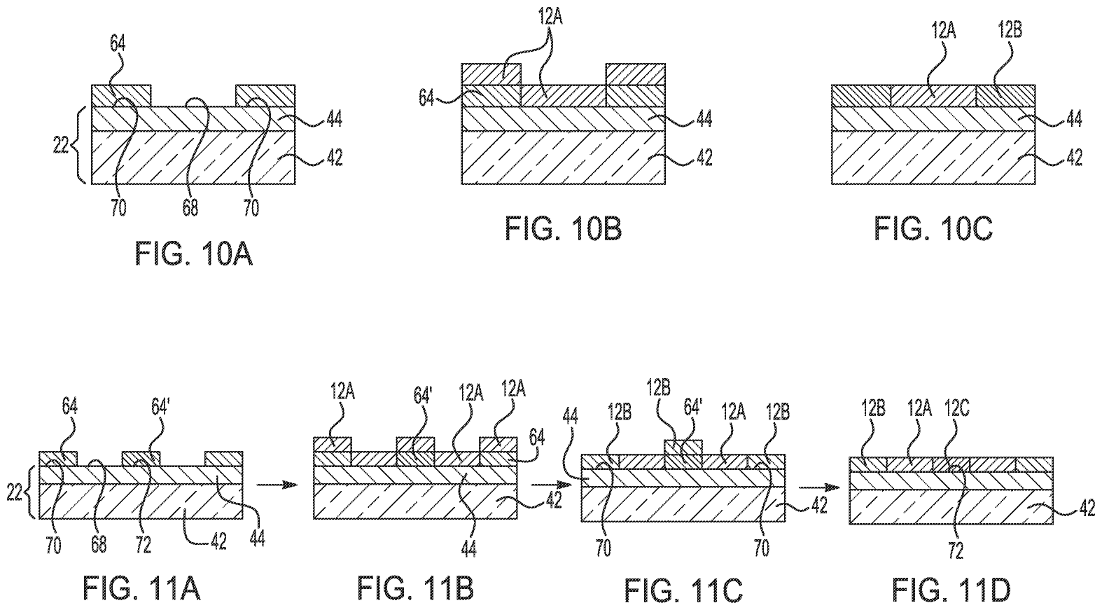

The example shown in FIG. 1E is similar to the example shown in FIG. 10, except that different types of cleavage sites 29, 31 are used in the linkers 33, 33′ attached to the cleavable primers 19′, 25′ of the respective primer subsets 13D, 15D. Examples of different cleavage sites 29, 31 that may be used in the respective linkers 33, 33′ attached to the cleavable primers 19′, 25′ include any combination of the following: vicinal diol, uracil, allyl ether, disulfide, restriction enzyme site, and 8-oxoguanine.

In still other examples, the flow cell includes different capture primers arranged in rows and offset columns across the substrate. The capture primers are orthogonal, in that they enable different template strands (i.e., library templates) to be seeded at respective areas across the substrate surface. In an example, the capture primers are PX primers. The PX primers described herein may be used as capture primers that seed a library template molecule, but that do not otherwise participate in amplification as they are orthogonal to all of the other primers. For sequential paired end sequencing using the primers sets 14A, 14B, 14C, different PX primers may be included with the different primer sets 14A, 14B, 14C to capture different library template molecules.

The PX capture primers may be:

PX 5′→3′
(SEQ. ID. NO. 13)
AGGAGGAGGAGGAGGAGGAGGAGG
cPX (PX′) 5′→3′
(SEQ. ID. NO. 14)
CCTCCTCCTCCTCCTCCTCCTCCT

Each of the primers disclosed herein may also include a polyT sequence at the 5′ end of the primer sequence. In some examples, the polyT region includes from 2 T bases to 20 T bases. As specific examples, the polyT region may include 3, 4, 5, 6, 7, or 10 T bases.

Any of the primers 16A, 18A or 16B, 18B, or 16C, 18C (including the capture primers) may be terminated, at the 5′ end, with a functional group that is capable of single point covalent attachment with a functional group of the polymeric hydrogel 12A, 12B, 12C. Examples of terminated primers that may be used include an alkyne terminated primer, a tetrazine terminated primer, an azido terminated primer, an amino terminated primer, an epoxy or glycidyl terminated primer, a thiophosphate terminated primer, a thiol terminated primer, an aldehyde terminated primer, a hydrazine terminated primer, a phosphoramidite terminated primer, and a triazolinedione terminated primer. In one example, the primers are terminated with hexynyl. In some specific examples, a succinimidyl (NHS) ester terminated primer may be reacted with an amine of the polymeric hydrogel 12A, 12B, 12C, an aldehyde terminated primer may be reacted with a hydrazine of the polymeric hydrogel 12A, 12B, 12C, or an alkyne terminated primer may be reacted with an azide of the polymeric hydrogel 12A, 12B, 12C, or an azide terminated primer may be reacted with an alkyne or DBCO (dibenzocyclooctyne) of the polymeric hydrogel 12A, 12B, 12C, or an amino terminated primer may be reacted with an activated carboxylate group or NHS ester of the polymeric hydrogel 12A, 12B, 12C, or a thiol terminated primer may be reacted with an alkylating reactant (e.g., iodoacetamine or maleimide) of the polymeric hydrogel 12A, 12B, 12C, or a phosphoramidite terminated primer may be reacted with a thioether of the polymeric hydrogel 12A, 12B, 12C. While several examples have been provided, it is to be understood that any functional group that can be attached to the primer 16A, 18A or 16B, 18B, or 16C, 18C and/or the capture primers and that can attach to a functional group of the polymeric hydrogel 12A, 12B, 12C may be used. Similar functional group may be included at the 5′ end of any of the primers 17, 17′, 19, 19′, 25, 25′, 27, 27′.

Polymeric Hydrogel

In any of the examples set forth herein, the primer set(s) 14A, 14B, 14C is/are attached to a polymeric hydrogel 12A, 12B, 12C. The attachment of the primer set(s) 14A, 14B, 14C leaves a template-specific portion of the primers 16A, 18A, or 16B, 18B, or 16C, 18C free to anneal to its cognate template and the 3′ hydroxyl group free for primer extension. It is to be understood that in the examples disclosed herein, the primer subset 13A, 15A or 13B, 15B, or 13C, 15C, or 13D, 15D may be used instead of one of the primer set(s) 14A, 14B, 14C. In these instances, the particular reactive area 10A, 10B, or 10C would include the primers 17, 19, 25, 27 or 17′, 19′, 25′, 27′ attached to the polymeric hydrogel 12A, 12B, or 12C (in the manner described in reference to FIG. 1B, FIG. 1C, FIG. 1D, or FIG. 1E).

In some examples, the polymeric hydrogel 12A, 12B, 12C is the same in each of the reactive areas 10A, 10B, 10C. In these examples, the polymeric hydrogel 12A, 12B, 12C is chemically the same, and any technique disclosed herein may be used to sequentially attach the primers 16A, 18A, or 16B, 18B, or 16C, 18C of the respective sets 14A, 14B, 14C to the respective reactive areas 10A, 10B, 10C. When the primer subset 13A, 15A or 13B, 15B, or 13C, 15C, or 13D, 15D is used in one of the areas 10A, 10B, 10C, the primers 17, 19 or 17′ 19′ and 25, 27 or 25′, 27′ of the subsets 13A, 15A or 13B, 15B, or 13C, 15C, or 13D, 15D are sequentially attached using the example techniques disclosed herein.

In other examples, the polymeric hydrogel 12A, 12B, 12C is different in each of the reactive areas 10A, 10B, 10C. For example, the polymeric hydrogel 12A, 12B, 12C in each of the reactive areas 10A, 10B, 10C may include different functional groups that are able to attach to the terminal functional group of the respective primers 16A, 18A, or 16B, 18B, or 16C, 18C. When the primer subset 13A, 15A or 13B, 15B, or 13C, 15C, or 13D, 15D is used in one of the areas 10A, 10B, 10C, the polymeric hydrogel regions 12A1, 12A2 include respective functional groups for attaching the primers 17, 19 or 17′ 19′ and 25, 27 or 25′, 27′ of the particular subset 13A, 15A or 13B, 15B, or 13C, 15C, or 13D, 15D. In some instances, the different functional groups are functional groups that have been introduced to the polymeric hydrogel that is deposited on the substrate.

The polymeric hydrogel 12A, 12B, 12C may be any gel material that can swell when liquid is taken up and can contract when liquid is removed, e.g., by drying. In an example, the polymeric hydrogel includes an acrylamide copolymer. Some examples of the acrylamide copolymer are represented by the following structure (I):

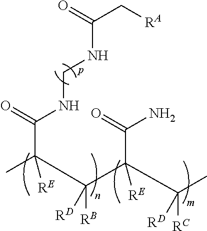

wherein:

R^{A} is selected from the group consisting of azido, optionally substituted amino, optionally substituted alkenyl, optionally substituted alkyne, halogen, optionally substituted hydrazone, optionally substituted hydrazine, carboxyl, hydroxy, optionally substituted tetrazole, optionally substituted tetrazine, nitrile oxide, nitrone, sulfate, and thiol;

R^{B} is H or optionally substituted alkyl;

R^{C}, R^{D}, and R^{E} are each independently selected from the group consisting of H and optionally substituted alkyl;

each of the —(CH₂)ₚ— can be optionally substituted;

p is an integer in the range of 1 to 50;

n is an integer in the range of 1 to 50,000; and

m is an integer in the range of 1 to 100,000.

One specific example of the acrylamide copolymer represented by structure (I) is poly(N-(5-azidoacetamidylpentyl)acrylamide-co-acrylamide, PAZAM.

One of ordinary skill in the art will recognize that the arrangement of the recurring “n” and “m” features in structure (I) are representative, and the monomeric subunits may be present in any order in the polymer structure (e.g., random, block, patterned, or a combination thereof).

The molecular weight of the acrylamide copolymer may range from about 5 kDa to about 1500 kDa or from about 10 kDa to about 1000 kDa, or may be, in a specific example, about 312 kDa.

In some examples, the acrylamide copolymer is a linear polymer. In some other examples, the acrylamide copolymer is a lightly cross-linked polymer.

In other examples, the gel material may be a variation of structure (I). In one example, the acrylamide unit may be replaced with N,N-dimethylacrylamide

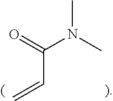

In this example, the acrylamide unit in structure (I) may be replaced with

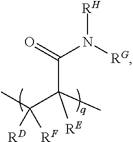

where R^{D}, R^{E}, and R^{F} are each H or a C1-C6 alkyl, and R^{G} and R^{H} are each a C1-C6 alkyl (instead of H as is the case with the acrylamide). In this example, q may be an integer in the range of 1 to 100,000. In another example, the N,N-dimethylacrylamide may be used in addition to the acrylamide unit. In this example, structure (I) may include

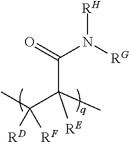

in addition to the recurring “n” and “m” features, where R^{D}, R^{E}, and R^{F} are each H or a C1-C6 alkyl, and R^{G} and R^{H} are each a C1-C6 alkyl. In this example, q may be an integer in the range of 1 to 100,000.

As another example of the polymeric hydrogel 12A, 12B, 12C, the recurring “n” feature in structure (I) may be replaced with a monomer including a heterocyclic azido group having structure (II):

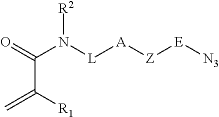

wherein R¹ is H or a C1-C6 alkyl; R₂ is H or a C1-C6 alkyl; L is a linker including a linear chain with 2 to 20 atoms selected from the group consisting of carbon, oxygen, and nitrogen and 10 optional substituents on the carbon and any nitrogen atoms in the chain; E is a linear chain including 1 to 4 atoms selected from the group consisting of carbon, oxygen and nitrogen, and optional substituents on the carbon and any nitrogen atoms in the chain; A is an N substituted amide with an H or a C1-C4 alkyl attached to the N; and Z is a nitrogen containing heterocycle. Examples of Z include 5 to 10 carbon-containing ring members present as a single cyclic structure or a fused structure. Some specific examples of Z include pyrrolidinyl, pyridinyl, or pyrimidinyl. As still another example, the polymeric hydrogel 12A, 12B, 12C may include a recurring unit of each of structure (III) and (IV):

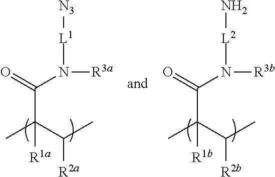

wherein each of R^{1a}R^{2a}, R^{1b} and R^{2b} is independently selected from hydrogen, an optionally substituted alkyl or optionally substituted phenyl; each of R^{1a} and R^{2b} is independently selected from hydrogen, an optionally substituted alkyl, an optionally substituted phenyl, or an optionally substituted C7-C14 aralkyl; and each L¹ and L² is independently selected from an optionally substituted alkylene linker or an optionally substituted heteroalkylene linker.

In still another example, the acrylamide copolymer is formed using nitroxide mediated polymerization, and thus at least some of the copolymer chains have an alkoxyamine end group. In the copolymer chain, the term “alkoxyamine end group” refers to the dormant species —ONR₁R₂, where each of R₁ and R₂ may be the same or different, and may independently be a linear or branched alkyl, or a ring structure, and where the oxygen atom is attached to the rest of the copolymer chain. In some examples, the alkoxyamine may also be introduced into some of the recurring acrylamide monomers, e.g., at position R^{A} in structure (I). As such, in one example, structure (I) includes an alkoxyamine end group; and in another example, structure (I) includes an alkoxyamine end group and alkoxyamine groups in at least some of the side chains.

It is to be understood that other molecules may be used to form the polymeric hydrogel 12A, 12B, 12C, as long as they are functionalized to graft one of the oligonucleotide primer sets 14A, 14B, 14C or subsets 13A, 15A or 13B, 15B or 13C, 15C or 13D, 15D thereto. Some examples of suitable polymeric hydrogel 12A, 12B, 12C materials include functionalized silanes, such as norbornene silane, azido silane, alkyne functionalized silane, amine functionalized silane, maleimide silane, or any other silane having functional groups that can attach the respective primer set 14A, 14B, 14C or subset 13A, 15A or 13B, 15B or 13C, 15C or 13D, 15D. Other examples of suitable polymeric hydrogel 12A, 12B, 12C materials include those having a colloidal structure, such as agarose; or a polymer mesh structure, such as gelatin; or a cross-linked polymer structure, such as polyacrylamide polymers and copolymers, silane free acrylamide (SFA), or an azidolyzed version of SFA. Examples of suitable polyacrylamide polymers may be synthesized from acrylamide and an acrylic acid or an acrylic acid containing a vinyl group, or from monomers that form [2+2] photo-cycloaddition reactions. Still other examples of suitable polymeric hydrogel 12A, 12B, 12C materials include mixed copolymers of acrylamides and acrylates. A variety of polymer architectures containing acrylic monomers (e.g., acrylamides, acrylates etc.) may be utilized in the examples disclosed herein, such as branched polymers, including dendrimers (e.g., multi-arm or star polymers), star-shaped or star-block polymers, and the like. For example, the monomers (e.g., acrylamide, acrylamide containing the catalyst, etc.) may be incorporated, either randomly or in block, into the branches (arms) of a dendrimer.

The polymeric hydrogel 12A, 12B, 12C may be formed using any suitable copolymerization process, such as nitroxide mediated polymerization (NMP), reversible addition-fragmentation chain-transfer (RAFT) polymerization, etc. The polymeric hydrogel 12A, 12B, 12C may also be deposited using any of the methods disclosed herein.

The attachment of the polymeric hydrogel 12A, 12B, 12C to the underlying substrate may be through covalent bonding. In some instances, the underlying substrate may first be activated, e.g., through silanization or plasma ashing. Covalent linking is helpful for maintaining the primer sets 14A, 14B, 14C and/or subsets 13A, 15A or 13B, 15B or 13C, 15C or 13D, 15D in the desired regions throughout the lifetime of the flow cell during a variety of uses.

Substrate

The substrate of the flow cell may be a single layer base support or a multi-layer structure upon which the reactive areas 10A, 10B, 10C are formed.

Examples of suitable single layer base supports include epoxy siloxane, glass, modified or functionalized glass, plastics (including acrylics, polystyrene and copolymers of styrene and other materials, polypropylene, polyethylene, polybutylene, polyurethanes, polytetrafluoroethylene (such as TEFLON® from Chemours), cyclic olefins/cyclo-olefin polymers (COP) (such as ZEONOR® from Zeon), polyimides, etc.), nylon (polyamides), ceramics/ceramic oxides, silica, fused silica, or silica-based materials, aluminum silicate, silicon and modified silicon (e.g., boron doped p+ silicon), silicon nitride (Si₃N₄), silicon oxide (SiO₂), tantalum pentoxide (Ta₂O₅) or other tantalum oxide(s) (TaOₓ), hafnium oxide (HfO₂), carbon, metals, inorganic glasses, or the like.

Examples of the multi-layered structure include the base support and at least one other layer thereon. Some examples of the multi-layered structure include glass or silicon as the base support, with a coating layer of tantalum oxide (e.g., tantalum pentoxide or another tantalum oxide(s) (TaOₓ)) or another ceramic oxide at the surface. Other examples of the multi-layered structure include the base support (e.g., glass, silicon, tantalum pentoxide, or any of the other base support materials) and a patterned resin as the coating layer. It is to be understood that any material that can be selectively deposited, or deposited and patterned to form depressions, regions of different heights, etc. may be used for the patterned resin.

In one example, the patterned resin is an inorganic oxide that may be selectively applied to the base support via vapor deposition, aerosol printing, or inkjet printing. Examples of suitable inorganic oxides include tantalum oxide (e.g., Ta₂O₅), aluminum oxide (e.g., Al₂O₃), silicon oxide (e.g., SiO₂), hafnium oxide (e.g., HfO₂), etc.

In another example, the patterned resin is a polymeric resin that may be applied to the base support and then patterned. Suitable deposition techniques include chemical vapor deposition, dip coating, dunk coating, spin coating, spray coating, puddle dispensing, ultrasonic spray coating, doctor blade coating, etc. Suitable patterning techniques include photolithography, nanoimprint lithography (NIL), stamping techniques, embossing techniques, molding techniques, microetching techniques, etc. Some examples of suitable resins include a polyhedral oligomeric silsesquioxane based resin (e.g., POSS® from Hybrid Plastics), a non-polyhedral oligomeric silsesquioxane epoxy resin, a poly(ethylene glycol) resin, a polyether resin (e.g., ring opened epoxies), an acrylic resin, an acrylate resin, a methacrylate resin, an amorphous fluoropolymer resin (e.g., CYTOP® from Bellex), and combinations thereof.

As used herein, the term “polyhedral oligomeric silsesquioxane” refers to a chemical composition that is a hybrid intermediate (e.g., RSiO_{1.5}) between that of silica (SiO₂) and silicone (R₂SiO). An example of polyhedral oligomeric silsesquioxane may be that described in Kehagias et al., Microelectronic Engineering 86 (2009), pp. 776-778, which is incorporated by reference in its entirety. In an example, the composition is an organosilicon compound with the chemical formula [RSiO_{3/2}]ₙ, where the R groups can be the same or different. Example R groups for polyhedral oligomeric silsesquioxane include epoxy, azide/azido, a thiol, a poly(ethylene glycol), a norbornene, a tetrazine, acrylates, and/or methacrylates, or further, for example, alkyl, aryl, alkoxy, and/or haloalkyl groups. The resin composition disclosed herein may include one or more different cage or core structures as monomeric units.

In an example, the substrate may be fabricated using a round wafer having a diameter ranging from about 2 mm to about 300 mm, or from a rectangular sheet or panel having its largest dimension up to about 10 feet (˜3 meters). In an example, the substrate is fabricated using a round wafer having a diameter ranging from about 200 mm to about 300 mm. In another example, a panel may be used that is a rectangular support, which has a greater surface area than a 300 mm round wafer. Wafers, panels and other large substrate materials may be diced into the individual flow cell substrates. In another example, the substrate is a die having a width ranging from about 0.1 mm to about 10 mm. While example dimensions have been provided, it is to be understood that a substrate material with any suitable dimensions may be used to fabricate the substrate.

Flow Cell Architecture

A top view of a flow cell 20 is shown in FIG. 2. The flow cell 20 may include two patterned structures bonded together or one patterned structure bonded to a lid. Different examples of the patterned structures of the flow cell 20 are shown in FIG. 3A through FIG. 7D. Between the two patterned structures or the one patterned structure and the lid is a flow channel 21. The example shown in FIG. 2 includes eight flow channels 21. While eight flow channels 21 are shown, it is to be understood that any number of flow channels 21 may be included in the flow cell 20 (e.g., a single flow channel 21, four flow channels 21, etc.). Each flow channel 21 may be isolated from another flow channel 21 so that fluid introduced into a flow channel 21 does not flow into adjacent flow channel(s) 21. Some examples of the fluids introduced into the flow channel 21 may introduce reaction components (e.g., DNA sample, polymerases, sequencing primers, labeled nucleotides, etc.), washing solutions, deblocking agents, etc.

The flow channel 21 is at least partially defined by a patterned structure. The patterned structure may include a substrate, such as a single layer base support or a multi-layered structure. FIG. 2 depicts a top view of the flow cell 20, and thus depicts the top of the substrate of one patterned structure (e.g., when the flow cell 20 includes a lid) or the bottom of the substrate of a second patterned structure (e.g., when the flow cell 20 includes two patterned structures whose surface chemistry face each other).

In an example, the flow channel 21 has a rectangular or substantially rectangular configuration. The length and width of the flow channel 21 may be selected so a portion of the substrate surrounds the flow channel 21 and is available for attachment to a lid (not shown) or another patterned structure.

The depth of the flow channel 21 can be as small as a monolayer thick when microcontact, aerosol, or inkjet printing is used to deposit a separate material that defines the flow channel 21 walls. For other examples, the depth of the flow channel 21 can be about 1 μm, about 10 μm, about 50 μm, about 100 μm, or more. In an example, the depth may range from about 10 μm to about 100 μm. In another example, the depth may range from about 10 μm to about 30 μm. In still another example, the depth is about 5 μm or less. It is to be understood that the depth of the flow channel 21 may be greater than, less than or between the values specified above.

FIG. 3A through FIG. 7D depict different examples of the patterned structures, and thus depict different examples of the architecture within the flow channel(s) 21 of the flow cell 20. The description of these figures refers to the primer sets 14A, 14B, 14C, but it is to be understood that any one the primer sets 14A, 14B, 14C described in these figures may be replaced with any one of the primer subsets 13A, 15A or 13B, 15B or 13C, 15C or 13D, 15D described herein.

Two examples of the patterned structure 23A, 23B of the flow cell 20 are shown in FIG. 3A and FIG. 3C, respectively. These examples of the patterned structure 23A, 23B of the flow cell 20 include a substrate 22; a plurality of reactive regions 24, 24′, 24″ extending along the substrate 22; and a non-reactive region 26, 26′ separating one of the plurality of reactive regions, e.g., 24, from an adjacent one of the plurality of reactive regions, e.g., 24′; wherein: each of the plurality of reactive regions 24, 24′, 24″ includes alternating first and second (reactive) areas 10A, 10B positioned along the reactive region 24, 24′, 24″; each of the first areas 10A includes a first primer set 14A and each of the second areas 10B includes a second primer set 14B that is different than the first primer set 14A; and i) adjacent first and second areas 10A, 10B directly abut each other (see FIG. 3C) or ii) the first areas 10A are positioned on protrusions 28 and the second areas 10B are positioned in depressions 30 adjacent to the protrusions 28 (FIG. 3A and FIG. 3B).

In FIG. 3A, the substrate 22 is a multi-layer structure, including a single layer base support 42, and another layer 44 positioned over the single layer base support 42. For the patterned structure 23A shown in FIG. 3A, the substrate 22 may alternatively be a single layer base support 42. In FIG. 3C, the substrate 22 is a single layer base support 42. For the patterned structure 23B shown in FIG. 3C, the substrate 22 may alternatively be a multi-layer structure.

In both FIG. 3A and FIG. 3C, the patterned structure 23A, 23B includes a plurality of reactive regions 24, 24′, 24″ extending along the substrate 22. In one example, the reactive regions 24, 24′, 24″ extend partially along the length of the substrate 22, and along the entire length of the flow channel 21. Each reactive region 24, 24′, 24′ includes alternating first and second (reactive) areas 10A, 10B. The pattern of alternating areas 10A, 10B extends along the length of each of the reactive regions 24, 24′, 24″.

In the example shown in FIG. 3A and FIG. 3B, the substrate 22 includes protrusions 28 and depressions 30 that alternate along the reactive regions 24, 24′, 24″. In this example, the protrusions 28 and depressions 30 are formed in the layer 44. These features 28, 30 are shown in the cross-sectional view in FIG. 3B. As depicted in FIG. 3A and FIG. 3B, respective active areas 10A are positioned over each of the protrusions 28 and respective active areas 10B are positioned in each of the depressions 30.

The protrusions 28 extend a first height H₁ from the bottom of the substrate 22 and the depressions 30 extend a second height H₂ from the bottom of the substrate 22, where the first height H₁ is greater than the second height H₂. The surface of each of the protrusions 28 may be coplanar with the surface of the non-reactive regions 26, 26′. In this example, the surface of the non-reactive regions 26, 26′ is also the surface of the substrate 22. The surface of each of the depressions 30 is positioned at a depth measured from the protrusion/non-reactive region/substrate surface (e.g., surface of layer 44). The depth is at least 150 nm. This depth is particularly suitable for library templates that are 500 base pairs (bp) long (e.g., assuming 0.35 nm/bp). The depth should not exceed a height (depth) to width ratio of about 10:1. In some examples, the depth can range from about 150 nm to about 100 μm, e.g., about 0.5 μm, about 1 μm, about 10 μm, or more.

The shape of the protrusions 28 and the depressions 30 in the x-y plane of the substrate 22 may be a square or a rectangle. The protrusions 28 and the depressions 30 may have the same shape, and thus the same x-y dimensions. Each of the length and the width can be about 150 nm or more. In an example, the length and the width may each range from about 150 nm to about 100 μm, e.g., about 0.5 μm, about 2 μm, about 10 μm, or more. The length and width may be selected so they do not exceed a length to width aspect ratio of about 10:1. The width of each of the protrusions 28 and the depressions 30 is equal to the width of the reactive regions 24, 24′, 24″.

The size of each protrusion 28 and depression 30 may be characterized by its surface and opening area, respectively. The surface area of the protrusion 28 and the opening area of the depression 30 may range from about 1×10⁻³ μm² to about 100 μm², e.g., about 1×10⁻² μm², about 0.1 μm², about 1 μm², at least about 10 μm², or more, or less.

The depression 30 may also be characterized by its volume. For example, the volume can range from about 1×10⁻³ μm³ to about 100 μm³, e.g., about 1×10⁻² μm³, about 0.1 μm³, about 1 μm³, about 10 μm³, or more, or less.

As shown in FIG. 3A, the alternating protrusions 28 and depressions 30 within each of the reactive regions 24, 24′, or 24″ directly abut one another. As such, the edge of one protrusion 28 is also the edge of one depression 30. In this example, the only exposed portion of the substrate 22 between the alternating protrusions 28 and depressions 30 within one of the reactive regions 24, 24′, or 24″ is a sidewall 32 (extending along the z axis) of the protrusion 28 and the depression 30 that does not have the polymeric hydrogel 12A, 12B or the primer sets 14A, 14B applied thereto. This sidewall 32 defines an interstitial region between the active areas 10A, 10B.

In the examples shown in FIG. 3A and FIG. 3B, the lack of an interstitial region in the x-y plane of the substrate 22 between the alternating protrusions 28 and depressions 30 decreases the average pitch of the active areas 10A, 10B across the substrate 22. In this example, the average pitch is the spacing from the center of one active area 10A to the center of an adjacent active area 10B (center-to-center spacing). The average pitch may range from about 50 nm to about 100 μm. In an example, the average pitch may range from about 50 nm to about 2 μm. In another example, the average pitch can be, for example, about 0.4 μm.

In this example, the active areas 10A, 10B are next to one another, but do not directly abut one another as one (10A) is positioned on the protrusion 28 and the other (10B) is positioned within the depression 30.

The active area 10A positioned over each of the protrusions 28 includes the polymeric hydrogel 12A and the primer set 14A. Thus, the x-y dimensions of each active area 10A are equal to the x-y dimensions of the protrusion 28 upon which the active area 10A is applied. The active area 10B positioned within each of the depressions 30 includes the polymeric hydrogel 12B and the primer set 14B. Thus, the x-y dimensions of each active area 10B are equal to the x-y dimensions of the depression 30 in which the active area 10B is applied.

As mentioned herein, the polymeric hydrogels 12A, 12B may be the same or may be different, and the primer sets 14A, 14B are different. In one example, each of the first and second areas 10A, 10B includes the same polymeric hydrogel 12A=12B to which the respective first and second primer sets 14A, 14B are attached. In another example, the first area 10A includes a first polymeric hydrogel 12A to which the first primer set 14A is attached; the second area 10B includes a second polymeric hydrogel 12B to which the second primer set 14B is attached; and the first polymeric hydrogel 12A and the second polymeric hydrogel 12B include orthogonal functional groups to respectively attach the first primer set 14A and the second primer set 14B.

Referring now to the example shown in FIG. 3C, the substrate 22 is planar, and thus does not include protrusions 28 or depressions 30. As such, the alternating active areas 10A, 10B within the respective reactive regions 24, 24′, or 24″ are positioned on the substrate surface.

In the example shown in FIG. 3C, the active areas 10A, 10B do directly abut one another. As such, the substrate surface is not exposed between the alternating active areas 10A, 10B, and thus there is no interstitial region between adjacent active areas 10A, 10B. The lack of an interstitial region in the x-y plane of the substrate 22 between the alternating active areas 10A, 10B decreases the average pitch of the active areas 10A, 10B across the substrate 22. In this example, the average pitch is the spacing from the center of one active area 10A to the center of an adjacent active area 10B (center-to-center spacing) in any one reactive region 24, 24′, 24″. The average pitch may range from about 50 nm to about 100 μm. In an example, the average pitch may range from about 50 nm to about 2 μm. In an example, the average pitch can be, for example, about 0.4 μm.

In this example, the shape of the active areas 10A, 10B in the x-y plane of the substrate 22 may be a square or a rectangle. The active areas 10A, 10B may have the same shape, and thus the same x-y dimensions. Each of the length and the width can range from about 150 nm to about 100 μm, e.g., about 0.5 μm, about 1 μm, about 10 μm, or more. The length and width may be selected so they do not exceed a length to width aspect ratio of about 10:1. The width of each of the active areas 10A, 10B is equal to the width of the reactive regions 24, 24′, 24″.

In the example shown in FIG. 3C, the active area 10A includes the polymeric hydrogel 12A and the primer set 14A, and the active area 10B includes the polymeric hydrogel 12B and the primer set 14B. As mentioned herein, the polymeric hydrogels 12A, 12B may be the same or may be different, and the primer sets 14A, 14B are different. In one example, each of the first and second areas 10A, 10B includes the same polymeric hydrogel 12A=12B to which the respective first and second primer sets 14A, 14B are attached. In another example, the first area 10A includes a first polymeric hydrogel 12A to which the first primer set 14A is attached; the second area 10B includes a second polymeric hydrogel 12B to which the second primer set 14B is attached; and the first polymeric hydrogel 12A and the second polymeric hydrogel 12B include orthogonal functional groups to respectively attach the first primer set 14A and the second primer set 14B.

In both of the examples shown in FIG. 3A and FIG. 3C, the reactive regions 24, 24′, 24″ are separated by respective non-reactive regions 26, 26′. The non-reactive regions 26, 26′ are regions of the substrate 22 that are free of the surface chemistry (e.g., the polymeric hydrogel 12A, 12B and the primer sets 14A, 14B) that is located in the active areas 10A, 10B. The non-reactive regions 26, 26′ extend in the same direction as the reactive regions 24, 24′, 24″ along the substrate 22. In the examples shown in FIG. 3A and FIG. 3C, the non-reactive regions 26, 26′ extend along the length of the substrate 22. As noted, one non-reactive region, e.g., 26, separates one reactive region, e.g., 24, from an immediately adjacent reactive region, e.g., 24′. The width of each non-reactive region 26, 26′ may be large enough to reduce or eliminate pad hopping between the active areas 10A, 10B in one reactive region, e.g., 24′, to the active areas 10A, 10B in an immediately adjacent reactive region, e.g., 24 and 24″. In an example, the width of the non-reactive region 26, 26′ can be 150 nm or more. In an example, the width may each range from about 150 nm to about 100 μm, e.g., about 0.5 μm, about 2 μm, about 10 μm, or more. In an example, the width of the non-reactive region 26, 26′ may be about 0.3 μm.

Other non-reactive regions 26, 26′ may also be located at the outermost surface of the substrate 22 (e.g., at one or more regions located at the perimeter). These regions 26, 26′ may be available for bonding to non-reactive regions 26, 26′ of another patterned structure 23A or 23B, or to a lid.

In the examples shown in FIG. 3A and FIG. 3C, the number of reactive regions 24, 24′, 24″ and the number of active areas 10A, 10B within each reactive region 24, 24′, 24″ will depend upon the length and width of the channel 21 in which the reactive and non-reactive regions are positioned, the width of each non-reactive region 26, 26′, and the x and y dimensions of active areas 10A, 10B. The lack of an interstitial region between the active areas 10A, 10B in the x-y plane of the substrate 22 should increase the density (number) of the active areas 10A, 10B in a defined area. In an example, the active areas 10A, 10B may be present at a density of approximately 3.6 million per mm².

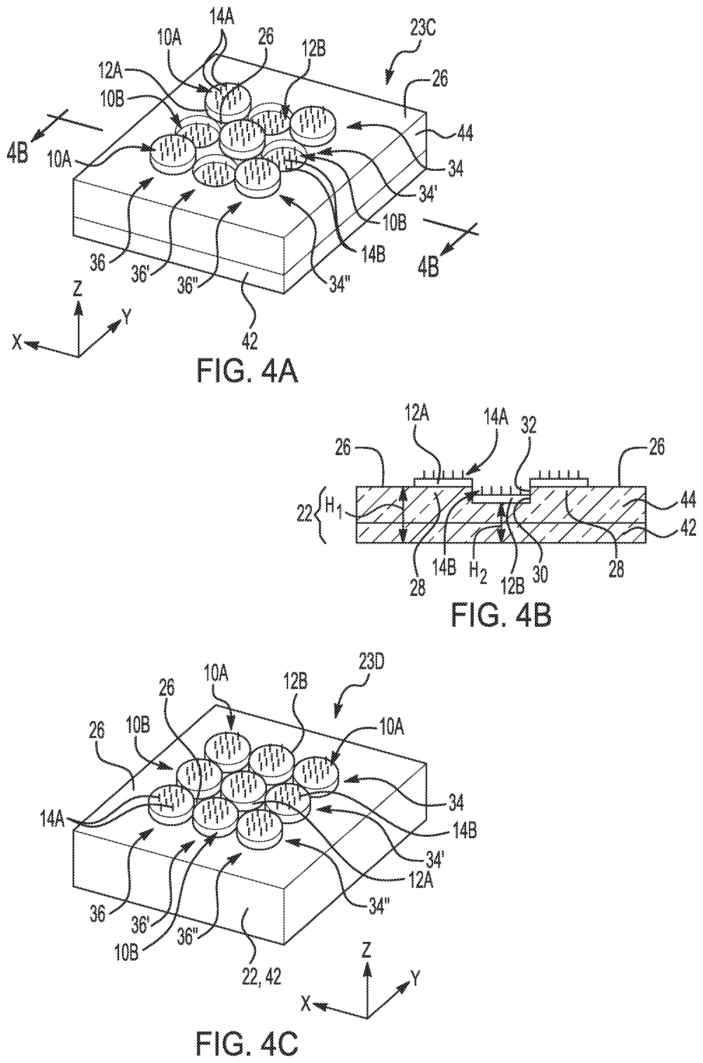

Two additional examples of the patterned structure 23C, 23D of the flow cell 20 are shown in FIG. 4A and FIG. 4B, respectively. These examples of the patterned structure 23C, 23D of the flow cell 20 include a substrate 22; and rows 34, 34′, 34″ and columns 36, 36′, 36″ of alternating first and second areas 10A, 10B; wherein: each of the first areas 10A includes a first primer set 14A and each of the second areas 10B includes a second primer set 14B that is different than the first primer set 14A; and i) adjacent first and second areas 10A, 10B directly abut each other (FIG. 4C) or ii) the first areas 10A are positioned on protrusions 28 and the second areas 10B are positioned in depressions 30 adjacent to the protrusions 28 (FIG. 4A and FIG. 4B).

In FIG. 4A, the substrate 22 is a multi-layer structure, including a single layer base support 42, and another layer 44 positioned over the single layer base support 42. For the patterned structure 23C shown in FIG. 4A, the substrate 22 may alternatively be a single layer base support 42. In FIG. 4C, the substrate 22 is a single layer base support 42. For the patterned structure 23B shown in FIG. 4C, the substrate 22 may alternatively be a multi-layer structure.

In both FIG. 4A and FIG. 4C, the patterned structure 23C, 23D includes rows 34, 34′, 34″ and columns 36, 36′, 36″ of alternating first and second areas 10A, 10B. In one example, the rows 34, 34′, 34″ extend partially along the width of the substrate 22, and along the entire width of the flow channel 21; and the columns 36, 36′, 36″ extend partially along the length of the substrate 22, and along the entire length of the flow channel 21. Each row 34, 34′, 34″ and each column 36, 36′, 36″ includes alternating first and second (reactive) areas 10A, 10B.

In the example shown in FIG. 4A, the substrate 22 (e.g., layer 44) includes protrusions 28 and depressions 30 that alternate along the rows 34, 34′, 34″ and along the columns 36, 36′, 36″. The features 28, 30 in one row 34″ are shown in the cross-sectional view of FIG. 4B. As depicted in FIG. 4A and FIG. 4B, respective active areas 10A are positioned over each of the protrusions 28 and respective active areas 10B are positioned in each of the depressions 30.

The protrusions 28 extend a first height H₁ from the bottom of the substrate 22 and the depressions 30 extend a second height H₂ from the bottom of the substrate 22, where the first height H₁ is greater than the second height H₂. The surface of each of the protrusions 28 may be coplanar with the surface of the substrate 22. The surface of each of the depressions 30 is positioned at a depth measured from the substrate surface (e.g., from the surface of layer 44). The depth can range from about 150 nm to about 100 μm, e.g., about 0.5 μm, about 1 μm, about 10 μm, or more.

In the example in FIG. 4A, the shape of the protrusions 28 and the depressions 30 in the x-y plane of the substrate 22 may be a circle, a diamond, or a rotated square. The protrusions 28 and the depressions 30 may have the same shape, and thus the same x-y dimensions. The diameter (of the circle), or each of the length and the width (of the diamond or rotated square) can range from about 150 nm to about 100 μm, e.g., about 0.5 μm, about 1 μm, about 10 μm, or more. The length and width may be selected so they do not exceed a length to width aspect ratio of about 10:1. The width of each of the protrusions 28 and the depressions 30 is equal to the width of the reactive regions 24, 24′, 24″.

The size of each protrusion 28 and depression 30 may be characterized by its surface and opening area, respectively, as described in reference to FIG. 3A. The depression 30 may also be characterized by its volume, as described in reference to FIG. 3A.

As shown in FIG. 4A, the alternating protrusions 28 and depressions 30 within each of the rows 34, 34′, 34″ directly abut one another, and the alternating protrusions 28 and depressions 30 within each of the columns 36, 36′, 36″ directly abut one another. As such, some of the protrusions 28 and depressions 30 share a sidewall 32. When the protrusions 28 and depressions 30 are circles, the sidewall 32 is along the circumferences of the respective circles. When the protrusions 28 and depressions 30 are diamonds or rotated squares, the sidewall 32 is along corners of the respective diamonds or rotated squares. The sidewall 32 (extending along the z axis) of the protrusion 28 and the depression 30 does not have the polymeric hydrogel 12A, 12B or the primer sets 14A, 14B applied thereto, and thus defines an interstitial region between the active areas 10A, 10B.

Because the alternating protrusions 28 and depressions 30 abut one another, there is no interstitial region directly between the abutting portions. The lack of an interstitial region in the x-y plane of the substrate 22 directly between the abutting portions of the protrusions 28 and depressions 30 decreases the average pitch of the active areas 10A, 10B across the substrate 22. In this example, the average pitch is the spacing from the center of one active area 10A to the center of an adjacent active area 10B in the same row 34, 34′, 34″ or column 36, 36′, 36″ (center-to-center spacing). The average pitch may range from about 50 nm to about 100 μm. In an example, the average pitch may range from about 50 nm to about 2 μm. In an example, the average pitch can be, for example, about 0.4 μm.

In this example, the active areas 10A, 10B are next to one another, but do not directly abut one another as one (10A) is positioned on the protrusion 28 and the other (10B) is positioned within the depression 30.

The active area 10A positioned over each of the protrusions 28 includes the polymeric hydrogel 12A and the primer set 14A. Thus, the x-y dimensions of each active area 10A are equal to the x-y dimensions of the protrusion 28 upon which the active area 10A is applied. The active area 10B positioned within each of the depressions 30 includes the polymeric hydrogel 12B and the primer set 14B. Thus, the x-y dimensions of each active area 10B are equal to the x-y dimensions of the depression 30 in which the active area 10B is applied.

As mentioned herein, the polymeric hydrogels 12A, 12B may be the same or may be different, and the primer sets 14A, 14B are different. In one example, each of the first and second areas 10A, 10B includes the same polymeric hydrogel 12A=12B to which the respective first and second primer sets 14A, 14B are attached. In another example, the first area 10A includes a first polymeric hydrogel 12A to which the first primer set 14A is attached; the second area 10B includes a second polymeric hydrogel 12B to which the second primer set 14B is attached; and the first polymeric hydrogel 12A and the second polymeric hydrogel 12B include orthogonal functional groups to respectively attach the first primer set 14A and the second primer set 14B.

Referring now to the example shown in FIG. 4C, the substrate 22 is planar, and thus does not include protrusions 28 or depressions 30. As such, the alternating active areas 10A, 10B within the rows 34, 34′, 34″ or columns 36, 36′, 36′ are positioned on the substrate surface.

In the example shown in FIG. 4C, the active areas 10A, 10B do directly abut one another. As such, the substrate surface is not exposed directly between the alternating active areas 10A, 10B, and thus there is no interstitial region between adjacent active areas 10A, 10B. The lack of an interstitial region in the x-y plane of the substrate 22 between the alternating active areas 10A, 10B decreases the average pitch of the active areas 10A, 10B across the substrate 22. In this example, the average pitch is the spacing from the center of one active area 10A to the center of an adjacent active area 10B (center-to-center spacing) in any one row 34, 34′, 34″ or column 36, 36′, 36″. The average pitch may range from about 50 nm to about 100 μm. In an example, the average pitch may range from about 50 nm to about 2 μm. In an example, the average pitch can be, for example, about 0.4 μm.

In this example, the shape of the active areas 10A, 10B in the x-y plane of the substrate 22 may be a circle, a diamond, or a rotated square. The active areas 10A, 10B may have the same shape, and thus the same x-y dimensions. The diameter (of the circle), or each of the length and the width (of the diamond or rotated square) can range from about 150 nm to about 100 μm, e.g., about 0.5 μm, about 1 μm, about 10 μm, or more.

In the example shown in FIG. 4C, the active area 10A includes the polymeric hydrogel 12A and the primer set 14A, and the active area 10B includes the polymeric hydrogel 12B and the primer set 14B. As mentioned herein, the polymeric hydrogels 12A, 12B may be the same or may be different, and the primer sets 14A, 14B are different. In one example, each of the first and second areas 10A, 10B includes the same polymeric hydrogel 12A=12B to which the respective first and second primer sets 14A, 14B are attached. In another example, the first area 10A includes a first polymeric hydrogel 12A to which the first primer set 14A is attached; the second area 10B includes a second polymeric hydrogel 12B to which the second primer set 14B is attached; and the first polymeric hydrogel 12A and the second polymeric hydrogel 12B include orthogonal functional groups to respectively attach the first primer set 14A and the second primer set 14B.

In both of the examples shown in FIG. 4A and FIG. 4C, a non-reactive region 26 is located at the intersection of four active areas 10A, 10B. These four active areas 10A, 10B are positioned in a square like configuration, such that each active region 10A is next to two different active regions 10B and each active region 10B is next to two different active regions 10A. The non-reactive region 26 separates the active regions 10A, 10B that are diagonal to one another in the square like configuration.

Other non-reactive regions 26 may also be located at the outermost surface (e.g., along the perimeter) of the substrate 22. These regions 26 may be available for bonding to non-reactive regions 26 of another patterned structure 23C or 23D, or to a lid.

In the examples shown in FIG. 4A and FIG. 4C, the number of rows 34, 34′, 34″, columns 36, 36′, 36″, and the number of active areas 10A, 10B within each row 34, 34′, 34″ and column 36, 36′, 36″ will depend upon the length and width of the channel 21 in which the active areas 10A, 10B are positioned, and the x and y dimensions of active areas 10A, 10B. The lack of an interstitial region directly between the active areas 10A, 10B in the x-y plane of the substrate 22 should increase the density (number) of the active areas 10A, 10B in a defined area. In an example, the active areas 10A, 10B may be present at a density of approximately 6.3 million per mm².

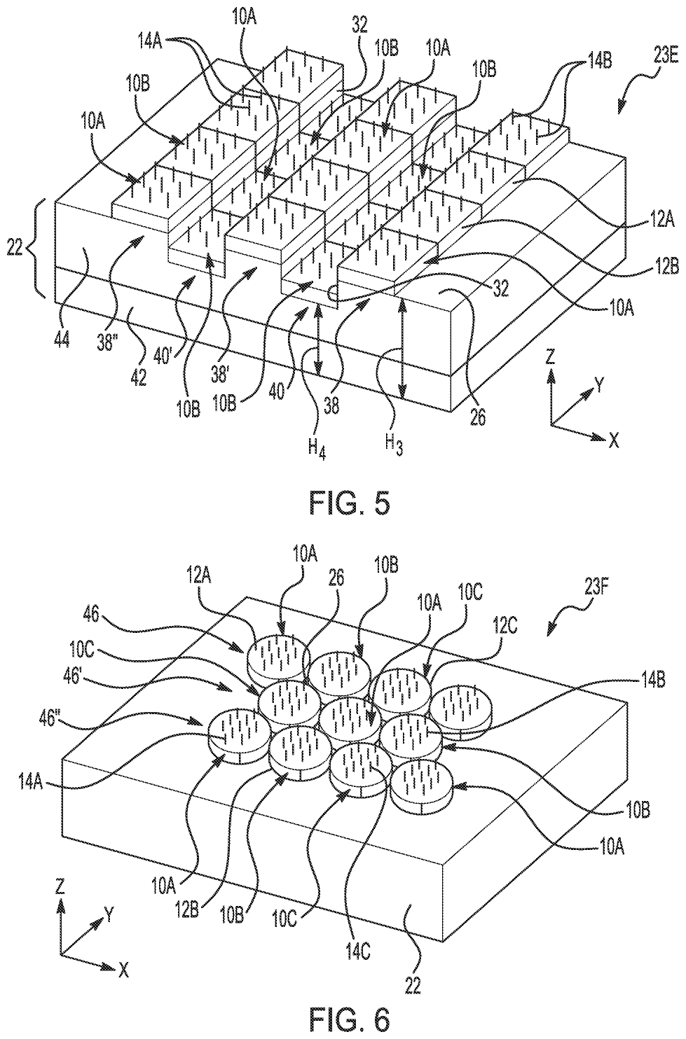

Still another example of the patterned structure 23E of the flow cell 20 is shown in FIG. 5. This example of the patterned structure 23E of the flow cell 20 include a substrate 22 having alternating regions 38, 38′, 38″ of a first height H₃ and regions 40, 40′ of a second height H₄; and alternating first and second areas 10A, 10B extending along the regions 38, 38′, 38″ of the first height H₃ and extending along the regions 40, 40′ of the second height H₄; wherein each of the first areas 10A includes a first primer set 14A, and each of the second areas 10B includes a second primer set 14B that is different than the first primer set 14A.

In FIG. 5, the substrate 22 is a multi-layer structure. As depicted, the substrate 22 includes a base support 42, and another layer 44 positioned over the base support 42. In this example of the patterned structure 23E, the substrate 22 may alternatively be a single layer base support.

In this example, the substrate 22, and in particular the layer 44, is patterned with alternating regions 38, 38′, 38″ of the first height H₃ and regions 40, 40′ of the second height H₄. The layer 44 may be any example of the patterned resin set forth herein, and may be patterned with the varying heights H₃, H₄ using any of the techniques set forth herein. In one example, the layer 44 is patterned such that the regions 38, 38′, 38″ and the regions 40, 40′ extend partially along the length of the substrate 22, and along the entire length of the flow channel 21.

The first and second heights H₃, H₄ are measured from the bottom of the substrate 22, and the first height H₃ is greater than the second height H₄. In some examples, each of the regions 38, 38′, 38″ has the same height H₃, and each of the regions 40, 40′ has the same height H₄. In other examples, each of the regions 38, 38′, 38″ has a different height H₃, and each of the regions 40, 40′ has a different height H₄, as long as the height of each of the regions 38, 38′, 38″ is greater than the height of each of the regions 40, 40′. In any of the examples, a difference between the first height H₃ and the second height H₄ is at least 150 nm.

The surface of each of the regions 38, 38′, 38″ may be coplanar with the substrate surface. The surface of each of the regions 40, 40′ is positioned at a depth measured from the substrate/regions 38, 38′, 38″ surface. The depth can range from at least 150 nm to about 100 μm, e.g., about 0.5 μm, about 1 μm, about 10 μm, or more.

As shown in FIG. 5, adjacent regions (e.g., 38 and 40, 40 and 38′, 38′ and 40′, and 40′ and 38″) share a sidewall 32. In this example, the only exposed portion of the substrate 22 between the adjacent regions (e.g., 38 and 40, 40 and 38′, 38′ and 40′, and 40′ and 38″) is the sidewall 32, which extends along the z axis. The respective sidewalls 32 do not have the polymeric hydrogel 12A, 12B or the primer sets 14A, 14B applied thereto, and thus the sidewalls 32 define an interstitial region between the active areas 10A, 10B positioned over the regions 38, 38′, 38″ and those positioned over the regions 40, 40′.

Each of the regions 38, 38′, 38″ and the regions 40, 40′ includes alternating first and second (reactive) areas 10A, 10B. In the example shown in FIG. 5, the alternating pattern of the areas 10A, 10B along each of the regions 38, 38′, 38″ is 10A, 10B, 10A, 10B, etc., and the alternating pattern of the areas 10A, 10B along each of the regions 40, 40′ is the opposite, e.g., 10B, 10A, 10B, etc. As such, the alternating pattern of the areas 10A, 10B is observed both along the length of each of the regions 38, 38′, 38″, 40, 40′ (e.g., in the y direction in FIG. 5), and also across the regions 38″, 40′, 38′, 40, 38 in a direction perpendicular to the length (e.g., in the x direction in FIG. 5).

In this example, the active areas 10A, 10B in each of the regions 38, 38′, 38″, 40, 40′ (e.g., in the y direction in FIG. 5) are next to one another and directly abut one another. As such, there is no interstitial region between the active areas 10A, 10B that are part of a particular region 38, 38′, 38″, 40, 40′. In contrast, the active areas 10A, 10B in adjacent regions (e.g., 38 and 40, 40 and 38′, 38′ and 40′, and 40′ and 38″) are next to one another but do not directly abut one another due to the variation in height H₃, H₄ between adjacent regions. As mentioned herein, the sidewall 32 functions as an interstitial region between the active areas 10A, 10B across the regions 38″, 40′, 38′, 40, 38.

In this example, the shape of the active areas 10A, 10B in the x-y plane of the substrate 22 may be a square or a rectangle. The active areas 10A, 10B may have the same shape, and thus the same x-y dimensions. Each of the length and the width can range from about 150 nm to about 100 μm, e.g., about 0.5 μm, about 1 μm, about 10 μm, or more. The width of each of the active areas 10A, 10B in the regions 38, 38′, 38″ is equal to the width of the respective region 38, 38′, 38″; and the width of each of the active areas 10A, 10B in the regions 40, 40′ is equal to the width of the respective region 40, 40′.

In the example shown in FIG. 5, the active area 10A includes the polymeric hydrogel 12A and the primer set 14A, and the active area 10B includes the polymeric hydrogel 12B and the primer set 14B. As mentioned herein, the polymeric hydrogels 12A, 12B may be the same or may be different, and the primer sets 14A, 14B are different. In one example, each of the first and second areas 10A, 10B includes the same polymeric hydrogel 12A=12B to which the respective first and second primer sets 14A, 14B are attached. In another example, the first area 10A includes a first polymeric hydrogel 12A to which the first primer set 14A is attached; the second area 10B includes a second polymeric hydrogel 12B to which the second primer set 14B is attached; and the first polymeric hydrogel 12A and the second polymeric hydrogel 12B include orthogonal functional groups to respectively attach the first primer set 14A and the second primer set 14B.

The patterned structure 23E shown in FIG. 5 may further include a non-reactive region 26 located at at least a portion of the perimeter of the substrate 22, wherein the non-reactive region 26 is an exposed portion of the substrate 22 (e.g., of layer 44). In the example shown in FIG. 5, the non-reactive regions 26 are at opposed edges of the substrate 22 and extend along the length. These non-reactive regions 26 may be used for bonding to another patterned structure 23E or a lid, e.g., in order to create the flow channel 21 (not shown in FIG. 5).

In any of the examples shown in FIG. 3A through FIG. 5, the positioning of the active areas 10A, 10B may be reversed. For example, in FIG. 3A or FIG. 4A, the active areas 10A may be formed in the depressions 30 and the active areas 10B may be formed on the protrusions 28.

Still another example of the patterned structure 23F of the flow cell 20 is shown in FIG. 6. This example of the patterned structure 23F of the flow cell 20 includes a substrate 22; a plurality of first areas 10A, each first area 10A including a first primer set 14A and being isolated from each other first area 10A; a plurality of second areas 10B, each second area 10B including a second primer set 14B and being isolated from each other second area 10B by at least one adjacent first area 10A and at least one adjacent third area 10C; and a plurality of the third areas 10C, each third area 10C including a third primer set 14C and being isolated from each other third area 10C by at least one adjacent first area 10A and at least one adjacent second area 10B.

In FIG. 6, the substrate 22 is a single layer base support. In this example of the patterned structure 23F, the substrate 22 may alternatively be a multi-layer structure.

In this example, the substrate 22 is planar, and thus does not include protrusions 28 or depressions 30 or regions 38, 40, etc. of differing heights H₃, H₄. As such, the active areas 10A, 10B, 10C are positioned on the substrate surface.

In this example, the shape of the active areas 10A, 10B, 10C in the x-y plane of the substrate 22 may be a circle. The active areas 10A, 10B may have the same shape, and thus the same x-y dimensions. The diameter can range from about 150 nm to about 100 μm, e.g., about 0.5 μm, about 1 μm, about 10 μm, or more.

The active areas 10A, 10B, 10C may be arranged in several rows 46, 46′, 46″ across the substrate 22. In the example shown in FIG. 6, every other row 46′ is slightly offset from its immediately adjacent row(s) 46, 46″ so the actives areas 10A, 10B, 10C in the slight offset row(s) 46′ are between two active areas 10A, 10B, 10C in the immediately adjacent row(s) 46, 46″. The offset positioning from one row 46, 46′, 46″ to the next row 46, 46′, 46″ allows for the active areas 10A to be in contact with active areas 10B and/or 10C but not with other active areas 10A, for each of the active areas 10B to be in contact with areas 10A and/or 10C but not with other active areas 10B, and for each of the active areas 10C to be in contact with areas 10A and/or 10B but not with other active areas 10C.

The active areas 10A, 10B, 10C within a given row 46, 46′, 46″ are next to each other and thus directly abut one another (e.g., in the x direction), such that there is no interstitial region between the active areas 10A, 10B, 10C within a given row 46, 46′, 46″. Each active area 10A, 10B, 10C in the offset row(s) 46′ may directly abut one or two active areas 10A, 10B, 10C in the immediately adjacent row(s) 46, 46″ at a diagonal. In the example shown in FIG. 6, each active area 10A, 10B, 10C in the offset row 46′ is at a 45° diagonal with respect to two of the active areas 10A, 10B, 10C in the row 46, and with respect to two of the active areas 10A, 10B, 10C in the row 46″. As one example, the active area 10C in the offset row 46′ and the two diagonal active areas 10A, 10B in the row 46 are positioned in a triangle like configuration, such that the active regions 10A, 10B are next to each other (in the row 46) and are each at a diagonal with respect to the active region 10C (in the row 46′). As another example, the active area 10A in the offset row 46′ and the two diagonal active areas 10B, 10C in the row 46″ are positioned in a triangle like configuration, such that the active regions 10B, 10C are next to each other (in the row 46″) and are each at a diagonal with respect to the active region 10A (in the row 46′).

The offset positioning from one row 46, 46′, 46″ to the next row 46, 46′, 46″ generates a non-reactive region 26 at the intersection of each triangle like configuration. Other non-reactive regions 26 may also be located at the outermost surface of the substrate 22 (e.g., at the perimeter). These regions 26 may be available for bonding to non-reactive regions 26 of another patterned structure 23F, or to a lid.

In the example shown in FIG. 6, the active area 10A includes the polymeric hydrogel 12A and the primer set 14A, the active area 10B includes the polymeric hydrogel 12B and the primer set 14B, and the active area 10C includes the polymeric hydrogel 12C and the primer set 14C. As mentioned herein, the polymeric hydrogels 12A, 12B, 12C may be the same or may be different, and the primer sets 14A, 14B, 14C are different. In an example, the first primer set 14A includes P5 and P7 primers; the second primer set 14B includes any combination of PX, PA, PB, PC and/or PD primers; and the third primer set 14C includes any combination of PX, PA, PB, PC and/or PD that is different from the second primer set 14B. Any of the primer sets 14A, 14B, 14C may also be replaced with primer subsets 13A, 15A, or 13B, 15B, etc. In one example, each of the first, second, and third areas 10A, 10B, 10C includes the same polymeric hydrogel 12A=12B=12C to which the respective first, second, and third primer sets 14A, 14B, 14C are attached. In another example, the first area 10A includes a first polymeric hydrogel 12A to which the first primer set 14A is attached; the second area 10B includes a second polymeric hydrogel 12B to which the second primer set 14B is attached; the third area 10C includes a third polymeric hydrogel 12C to which the third primer set 14B is attached; and the first polymeric hydrogel 12A, the second polymeric hydrogel 12B, and the third polymeric hydrogel 12C include orthogonal functional groups to respectively attach the first primer set 14A, the second primer set 14B, and the third primer set 14C.

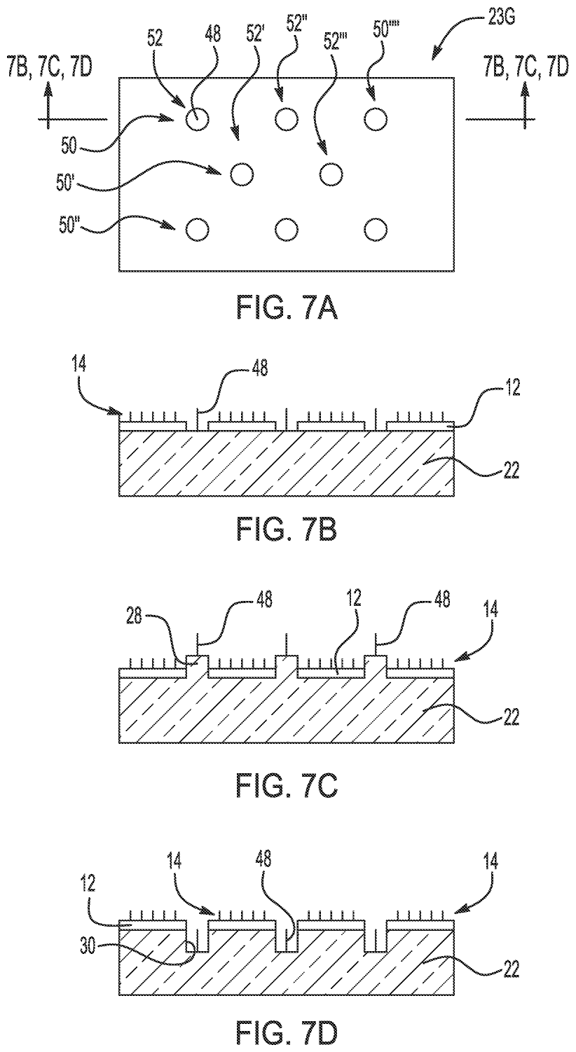

Still another example of the patterned structure 23G of the flow cell 20 is shown in FIG. 7A.

This example of the patterned structure 23G of the flow cell 20 include a substrate 22; a plurality of capture primers 48 arranged in rows 50, 50′, 50″, and offset columns 52, 52′, 52″, 52′″, 52″″ across the substrate 22; a continuous polymeric hydrogel 12 positioned over the substrate 22 and surrounding each of the plurality of capture primers 48; and a primer set 14 attached to the continuous polymeric hydrogel 12.

The PX primers set forth herein are suitable capture primers 48. Any of the primer sets or subsets set forth herein may be attached to the polymeric hydrogel 12 surrounding the capture primers 48. Each capture primer 48 hybridizes to one library template, which amplifies across the primer set 14. As the different clusters of amplicons physically collide, amplification ceases, creating the different active areas 10A, 10B, 10C.

Different configurations of the patterned structure 23G are shown in FIG. 7B through FIG. 7D. In FIG. 7B through FIG. 7D, the substrate 22 is a single layer base support. In any example of the patterned structure 23G, the substrate 22 may alternatively be a multi-layer structure.

In FIG. 7B, the substrate 22 is planar, and the plurality of capture primers 48 and the continuous polymeric hydrogel 12 are attached to the substrate surface.

In FIG. 7C, the substrate 22 includes protrusions 28 arranged in the rows 50, 50′, 50″ and the offset columns 52, 52′, 52″, 52′″, 52″″ across the substrate 22; and one of the plurality of capture primers 48 is positioned on each of the protrusions 28.

In FIG. 7D, the substrate 22 includes depressions 30 arranged in the rows 50, 50′, 50″ and the offset columns 52, 52′, 52″, 52′″, 52″″ across the substrate 22; and one of the plurality of capture primers 48 is positioned in each of the depressions 30.

In the examples shown in FIGS. 3A, 4A, 4C, 5, and 6, it is to be understood that active areas 10A, 10B, 10C that are formed on the substrate surface may instead be formed in depressions (e.g., 30). In these examples, the active areas 10A may be positioned in depressions 30 with a depth that is different from the depth of the depressions 30 containing active areas 10B, and different from the depth of the depressions 30 containing active areas 10C (if included). The difference in depth between the different depressions is at least 150 nm. When the various active areas 10A, 10B, 10C are formed in depressions with different depths (as opposed to some being formed on the substrate surface), the surface of the substrate can be cleaned of any polymeric hydrogel 12A, 12B, 12C, and primers 14A, 14B, etc. via polishing while the surface chemistry in the depressions remains intact.

Method for Making the Flow Cell Architectures

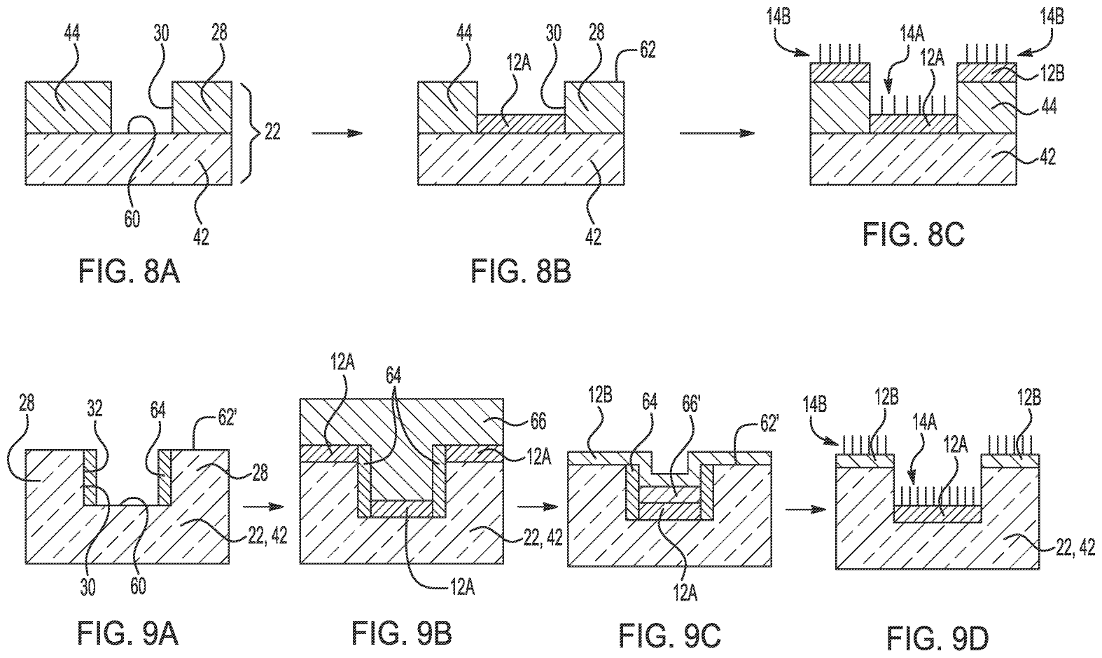

The architectures of the patterned structures 23A, 23C shown in FIG. 3A and FIG. 4A may be prepared by the method shown in FIG. 8A through FIG. 8C. With this example method, the patterned structures 23A, 23C include the multi-layered substrate 22. This method generally includes depositing a first polymeric hydrogel 12A on a multi-layered substrate (one example of substrate 22), the multi-layered substrate including a base support 42 including surface groups to attach to the first polymeric hydrogel 12A; a layer 44 positioned over the base support 42, the layer 44 including a material that is incapable of attaching to the first polymeric hydrogel 12A; and a plurality of depressions 30 defined in the layer 44 such that a portion 60 of the base support 42 is exposed at each of the plurality of depressions 30, whereby the first polymeric hydrogel 12A selectively attaches to the portion 60 of the base support 42 that is exposed at each of the plurality of depressions 30; activating the layer 44 with surface groups to attach a second polymeric hydrogel 12B; depositing the second polymeric hydrogel 12B such that it selectively attaches to the layer 44; grafting a first primer set 14A to the first polymeric hydrogel 12A; and grafting a second primer set 14B to the second polymeric hydrogel 14B, the second primer set 14B being different from the first primer set 14A.

While the first polymeric hydrogel 12A and primer set 14A are shown on the protrusions 28 in FIG. 3A and FIG. 4A, they are shown in the depressions 30 in FIG. 8C. Similarly, while the second polymeric hydrogel 12B and primer set 14B are shown in the depressions 30 in FIG. 3A and FIG. 4A, they are shown on the protrusions 28 in FIG. 8C. It is to be understood that the method shown in FIG. 8A through FIG. 8C may be performed such that the reactive areas 10A are formed in the depressions 30 and the reactive areas 10B are formed on the protrusions 28, or so that the reactive areas 10B are formed in the depressions 30 and the reactive areas 10A are formed on the protrusions 28.

As shown in FIG. 8A, the substrate 22 includes the base support 42 and a patterned layer 44 positioned thereon. In one example, the base support 42 is a resin material.

The layer 44 is formed over the base support 42. The layer 44 may be any material that can be selectively deposited, or deposited and patterned to form depressions 30 and protrusions 28. Suitable deposition techniques include chemical vapor deposition, dip coating, dunk coating, spin coating, spray coating, puddle dispensing, ultrasonic spray coating, doctor blade coating, aerosol printing, screen printing, microcontact printing, etc. Suitable patterning techniques include photolithography, nanoimprint lithography (NIL), stamping techniques, embossing techniques, molding techniques, microetching techniques, etc. Some examples of suitable resins include a polyhedral oligomeric silsesquioxane-based resin, a non-polyhedral oligomeric silsesquioxane epoxy resin, a poly(ethylene glycol) resin, a polyether resin (e.g., ring opened epoxies), an acrylic resin, an acrylate resin, a methacrylate resin, an amorphous fluoropolymer resin (e.g., CYTOP® from Bellex), and combinations thereof.

The arrangement of the depressions 30 and protrusions 28 may be as shown in FIG. 3A. As such, in one example, the layer 44 is patterned to include a plurality of first lines (similar to regions 24, 24′, 24″) extending along the layer 44, each of the first lines including some of the plurality of depressions 30 separated by non-patterned regions of the layer 44 (shown as protrusions 28 in FIG. 8A); and a second line (similar to regions 26, 26′) separating one of the plurality of first lines from an adjacent one of the plurality of first lines, the second line including a continuous non-patterned region of the layer 44 that extends a length of each of the plurality of first lines. The arrangement of the depressions 30 and protrusions 28 may alternatively be as shown in FIG. 4A. As such, in one example, the layer 44 is patterned to include rows (similar to rows 34, 34′, 34″) and columns (similar to columns 36, 36′, 36″) of alternating depressions 30 and non-patterned regions (shown as protrusions 28 in FIG. 8A) of the layer 44.

The layer 44 covers the base support 42 (as shown in FIG. 3A and FIG. 4A), except at the depressions 30 where a portion 60 of the activated based support 42 is exposed (as shown in FIG. 8A).

In one example, the exposed portions 60 of the base support 42 may be activated via plasma ashing to introduce surface groups that can attach to the first polymeric hydrogel 12A. The layer 44 may be masked during the plasma etching process so that it is not affected. In another example, the base support 42 may be exposed to activation (e.g., via plasma ashing or silanization) prior to the layer 44 being formed thereon. In either instance, the exposed portions 60 of the base support 42 are functionalized to selectively attach to the polymeric hydrogel 12A, and the layer 44 positioned over the base support 42 is incapable of attaching to the first polymeric hydrogel 12A.

As shown in FIG. 8B, the first polymeric hydrogel 12A is deposited on the multi-layered substrate 22 using any suitable deposition technique. In this example, the first polymeric hydrogel 12A selectively attaches to the exposed portion(s) 60 of the support 42, and not to the exposed portions of the layer 44.

The layer 44 is then cleaned (e.g., with NaOH) and activated with surface groups to attach a second polymeric hydrogel 12B. The process for activating the layer 44 may depend upon where it is desirable for the second polymeric hydrogel 12B to attach.

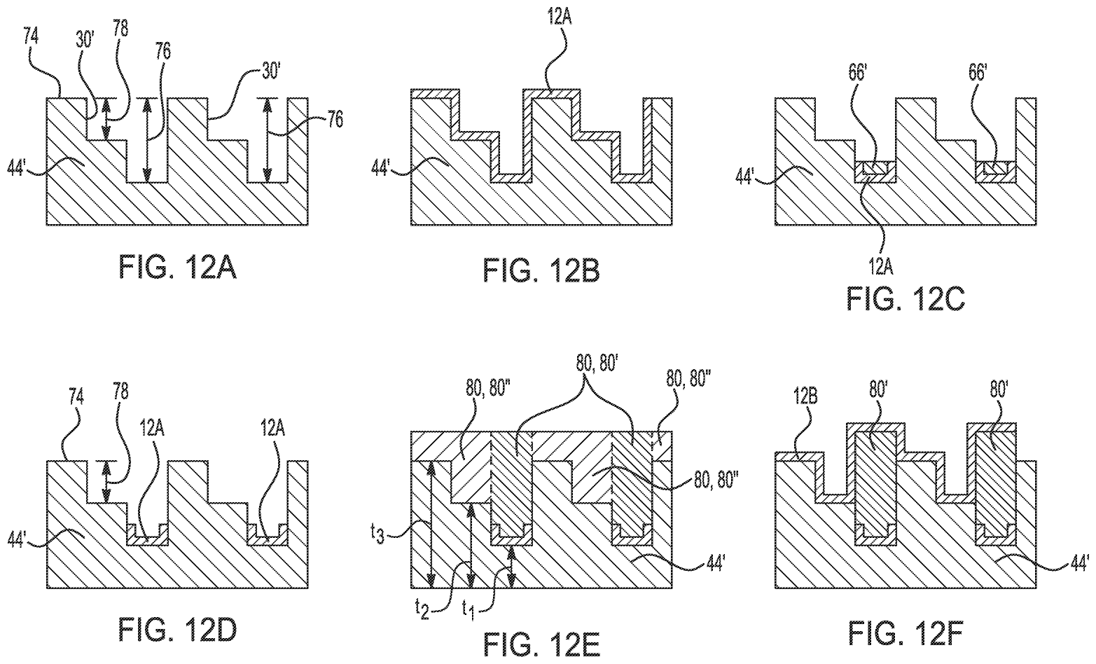

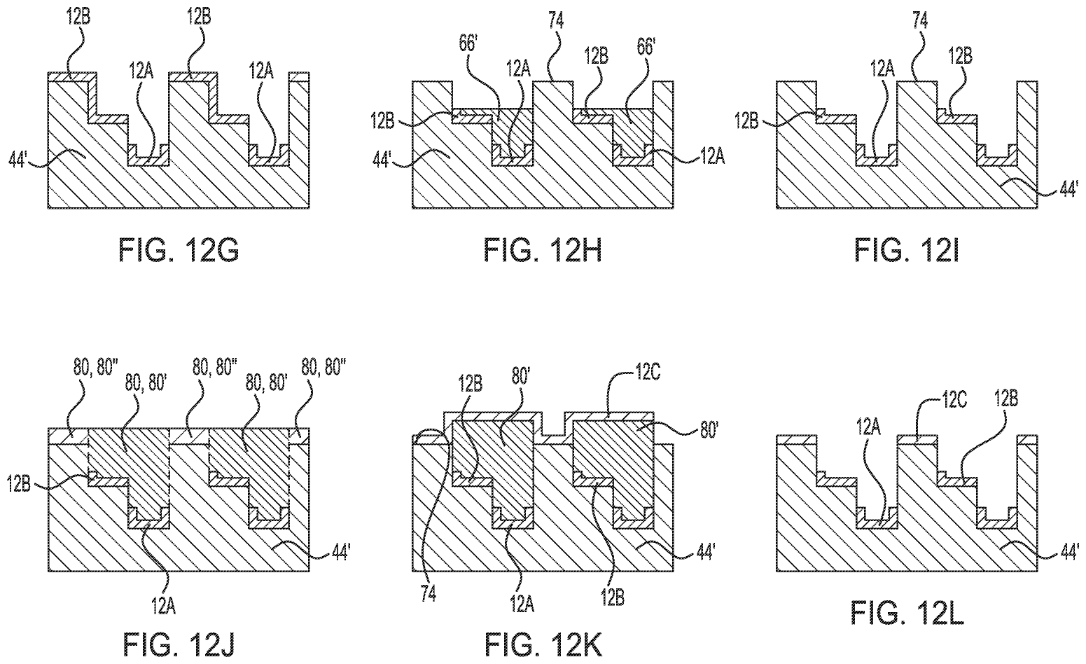

In the example shown in FIG. 3A, the non-reactive regions 26, 26′ do not have the polymeric hydrogels 12A, 12B applied thereto. Thus, it is not desirable to activate the continuous non-patterned regions of the layer 44 that form the non-reactive regions 26, 26′. The continuous non-patterned regions of the layer 44 (referred to as second line(s)) that ultimately form the non-reactive regions 26, 26′ may be masked (e.g., using a photoresist) during the activation of the layer 44 such that these line(s) are incapable of attaching to the second polymeric hydrogel 12B. In FIG. 3A, active areas are to be formed on the protrusions 28 that alternate along the (first) lines of the layer 44 with the depressions 28, and thus it is desirable to activate the protrusions 28. The protrusions 28 may be activated via silanization. Once the protrusions 28 are activated, the masking layer may be removed from the continuous non-patterned regions of the layer 44. The protrusions 28 contain surface groups to attach to the polymeric hydrogel (12A as shown in FIG. 3A or 12B as shown in FIG. 8C). In contrast, the continuous non-patterned regions of the layer 44 that form the non-reactive regions 26, 26′ do not have surface groups to attach the polymeric hydrogel (12A as shown in FIG. 3A or 12B as shown in FIG. 8C).

In the example shown in FIG. 4A, before the active areas (e.g., 10A) are formed over the protrusions 28, the depressions 30 are surrounded by non-patterned regions of the layer 44. Some of these non-patterned regions are shown in FIG. 8B at reference numeral 62. In this example, activating the layer 44 involves selectively silanizing the non-patterned regions (of the layer 44) to generate rows and columns of alternating depressions 30 (which have polymeric hydrogel 12A (FIG. 8B) or 12B (FIG. 4A) therein) and activated regions 62 of the layer 44, wherein the depressions 30 and the activated regions 62 are circular and have the same diameter. This activation process forms a pattern for the active areas that are to be formed on the protrusions 28.

The other polymeric hydrogel can then be applied, e.g., to the activated protrusions 28. In FIG. 8C, the other polymeric hydrogel is the second polymeric hydrogel 12B. In one example, the second polymeric hydrogel 12B attaches to the activated portions 62 on the protrusions 28. The second polymeric hydrogel 12B may be applied using any suitable deposition technique, and when the deposition is performed under high ionic strength (e.g., in the presence of 10×PBS, NaCl, KCl, etc.), the second polymeric hydrogel 12B does not deposit on or adhere to the first polymeric hydrogel 12A (i.e., the polymeric hydrogel in the depressions 30).

This method also includes attaching respective primer sets 14A, 14B to the polymeric hydrogels 12A, 12B. In some examples, the primer sets 14A, 14B may be pre-grafted to the respective polymeric hydrogels 12A, 12B. In these examples, additional primer grafting is not performed.

In other examples, the primer sets 14A, 14B are not pre-grafted to the respective polymeric hydrogels 12A, 12B. In these examples, the primer set 14A may be grafted after the polymeric hydrogel 12A is applied (e.g., at FIG. 8B). In these examples, the primer set 14B may be pre-grafted to the second polymeric hydrogel 12B. Alternatively, in these examples, the primer set 14B may not be pre-grafted to the second polymeric hydrogel 12B. Rather, the primer set 14B may be grafted after the second polymeric hydrogel 12B is applied (e.g., at FIG. 8C), as long as i) the second polymeric hydrogel 12B has different functional groups (than the first polymeric hydrogel 12A) for attaching the primer set 14B or ii) unreacted functional groups of the first polymeric hydrogel 12A have been quenched, e.g., using the Staudinger reduction to amines or additional click reaction with a passive molecule such as hexynoic acid.

When grafting is performed during the method, grafting may be accomplished using any suitable grafting techniques. As examples, grafting may be accomplished by flow through deposition (e.g., using a temporarily bound lid), dunk coating, spray coating, puddle dispensing, or by another suitable method. Each of these example techniques may utilize a primer solution or mixture, which may include the primer set 14A or 14B, water, a buffer, and a catalyst. With any of the grafting methods, the primer set 14A or 14B attaches to the reactive groups of the polymeric hydrogel 12A or 12B, and have no affinity for the other layers.

The architectures of the patterned structures 23A, 23C shown in FIG. 3A and FIG. 4A may also be prepared by the method shown in FIG. 9A through FIG. 9D. In this example method, the patterned structures 23A, 23C include the single layer base support 42. This method generally includes depositing a mask material 64 on sidewalls 32 of each depression 30 defined in a substrate 22; depositing a first polymeric hydrogel 12A on the substrate 22, whereby the first polymeric hydrogel 12A selectively attaches to portions 60 of the substrate 22 that are exposed at each of the plurality of depressions 30 and at regions 62′ separating the plurality of depressions 30; depositing a photoresist 66 on the substrate 22; etching to remove a first portion of the photoresist 66 and a first portion of the first polymeric hydrogel 12A from the regions 62′, whereby a second portion 66′ of the photoresist 66 and a second portion of the first polymeric hydrogel 12A remain in each of the plurality of depressions 30; activating the regions 62′ with surface groups to attach a second polymeric hydrogel 12B; depositing the second polymeric hydrogel 12B such that it selectively attaches to the regions 62′; removing the mask material 64 and the second portion of the photoresist 66; grafting a first primer set 14A to the first polymeric hydrogel 12A; and grafting a second primer set 14B to the second polymeric hydrogel 12B, the second primer set 14B being different from the first primer set 114A.

While the first polymeric hydrogel 12A and primer set 14A are shown on the protrusions 28 in FIG. 3A and FIG. 4A, they are shown in the depressions 30 in FIG. 9D. Similarly, while the second polymeric hydrogel 12B and primer set 14B are shown in the depressions 30 in FIG. 3A and FIG. 4A, they are shown on the protrusions 28 in FIG. 9D. It is to be understood that the method shown in FIG. 9A through FIG. 9D may be performed such that the reactive areas 10A are formed in the depressions 30 and the reactive areas 10B are formed on the protrusions 28, or so that the reactive areas 10B are formed in the depressions 30 and the reactive areas 10A are formed on the protrusions 28.

In the example shown in FIG. 9A, the depressions 30 are defined in a single layer base support 42. In this example, a multi-layer substrate could be used. If the multi-layer substrate were used, the depressions 30 would be formed in the outermost (top) layer such that any underlying layer would not be exposed. As such, whether the single layer base support 42 or a multi-layer substrate is used, the exposed portion 60 in the depressions 30 and the regions 62′ have the same surface groups.

The depressions 30 may be formed in the single layer base support 42 using any suitable technique, such as photolithography, nanoimprint lithography (NIL), stamping techniques, laser-assisted direct imprinting (LADI) embossing techniques, molding techniques, microetching techniques, etc. The technique used will depend, in part, upon the type of material used. As examples, the depressions 30 may be microetched into a glass single layer substrate or nanoimprinted into a nanoimprint lithography resin. When the multi-layer substrate is used, the depressions 30 may be formed in the outermost (top) layer using any suitable technique, such as nanoimprint lithography (NIL) or photolithography, etc. The technique used will depend, in part, upon the type of material used and will be performed such that any underlying layers are not exposed at the bottom of the depressions 30.

The arrangement of the depressions 30 and protrusions 28 may be as shown in FIG. 3A. As such, in one example, the single layer base support 42 is patterned to include a plurality of first lines (similar to regions 24, 24′, 24″) extending along the single layer base support 42, each of the first lines including some of the plurality of depressions 30 separated by non-patterned regions of the single layer base support 42 (shown as protrusions 28 in FIG. 9A); and a second line (similar to regions 26, 26′) separating one of the plurality of first lines from an adjacent one of the plurality of first lines, the second line including a continuous non-patterned region of the single layer base support 42 that extends a length of each of the plurality of first lines. The arrangement of the depressions 30 and protrusions 28 may alternatively be as shown in FIG. 4A. As such, in one example, the single layer base support 42 is patterned to include rows (similar to rows 34, 34′, 34″) and columns (similar to columns 36, 36′, 36″) of alternating depressions 30 and non-patterned regions (shown as protrusions 28 in FIG. 9A) of the single layer base support 42.

This example method continues with the application of a mask material 64 on sidewalls 32 of each depression 30. This is depicted in FIG. 9B. Examples of suitable materials for the mask material 64 include semi-metals, such as silicon, or metals, such as aluminum, copper, titanium, gold, silver, etc., or negative or positive photoresists. In some examples, the semi-metal or metal may be at least substantially pure (<99% pure). In other examples, molecules or compounds of the listed elements may be used, as they provide the desired etch stop or other function in a particular method. For example, oxides of any of the listed semi-metals (e.g., silicon dioxide) or metals (e.g., aluminum oxide) may be used, alone or in combination with the listed semi-metal or metal.

The mask material 64 may be applied to the sidewalls 32 using a photolithography process combined with either a lift-off technique or an etching technique. In other examples, selective deposition techniques, such as chemical vapor deposition (CVD) and variations thereof (e.g., low-pressure CVD or LPCVD), atomic layer deposition (ALD), and angled deposition, may be used to deposit the mask material 64 in the desirable areas. Alternatively, the mask material 64 may be applied across the substrate 22, and then selectively removed (e.g., via masking and etching) from the portions 60 and the regions 62′ to define the pattern on the sidewalls 32.

The single layer base support 42 may then be activated via plasma ashing. Plasma ashing introduces surface groups for attaching the polymeric hydrogel 12A or 12B to the exposed portions 60 and regions 62′ of the single layer base support 42.

As shown in FIG. 9B, the first polymeric hydrogel 12A is deposited on the single layer base support 42 using any suitable deposition technique. In this example, the first polymeric hydrogel 12A selectively attaches to the exposed portion(s) 60 and the regions 62′ of the single layer base support 42, and not to the mask material 64.

Also as shown in FIG. 9B, a photoresist 66 is applied to the single layer base support 42 over the mask 64 and the first polymeric hydrogel 12A. In this example, the entire photoresist 66 may be developed to form an insoluble portion, so that it can be exposed to an etching process.

In one example, the photoresist 66 is a negative photoresist (exposed region is insoluble in the developer). An example of suitable negative photoresist includes the NR® series photoresist (available from Futurrex). Other suitable negative photoresists include the SU-8 Series and the KMPR® Series (both of which are available from Kayaku Advanced Materials, Inc.), or the UVN™ Series (available from DuPont). When the negative photoresist is used, it is selectively exposed to certain wavelengths of light to render it insoluble. In these examples, a developer solution is not utilized as there are no soluble portions. In another example, the photoresist 66 is a positive photoresist (exposed region becomes soluble in the developer). Examples of suitable positive photoresists include the MICROPOSIT® S1800 series or the AZ® 1500 series, both of which are available from Kayaku Advanced Materials, Inc. Another example of a suitable positive photoresist is SPR™-220 (from DuPont). When the positive photoresist is used, it is not exposed to certain wavelengths of light to form a soluble region as it is desirable for the entire photoresist 66 to be insoluble.

A timed dry etching process may then be used to remove portions of the photoresist 66 and the first polymeric hydrogel 12A from the regions 62′ of the single layer base support 42. As shown in FIG. 9C, the timed dry etching is stopped so that a portion of the polymeric hydrogel 12A and a portion 66′ of the photoresist 66 remain in the depression 30. In one example, the timed dry etch may involve a reactive ion etch (e.g., with CF₄) where the photoresist 66 is etched at a rate of about 17 nm/min. In another example, the timed dry etch may involve a 100% O₂ plasma etch where the photoresist 66 is etched at a rate of about 98 nm/min.

As mentioned, during the etching of the photoresist 66, the polymeric hydrogel 12A over the regions 62′ may also be removed. A combustion reaction may be taking place, where the polymeric hydrogel 12A is converted to carbon dioxide and water and is evacuated from the etching chamber.

The regions 62′ of the single layer base support 42 may then be activated with surface groups to attach a second polymeric hydrogel 12B. The process for activating the single layer base support 42 may depend upon where it is desirable for the second polymeric hydrogel 12B to attach.

In the example shown in FIG. 3A, the non-reactive regions 26, 26′ do not have the polymeric hydrogels 12A, 12B applied thereto. Thus, it is not desirable to activate the continuous non-patterned regions of the single layer base support 42 that form the non-reactive regions 26, 26′. The continuous non-patterned regions of the single layer base support 42 (referred to as second line(s)) that ultimately form the non-reactive regions 26, 26′ may be masked, e.g., with mask material 64, during the activation of the single layer base support 42 such that these line(s) are incapable of attaching to the second polymeric hydrogel 12B. As such, the mask material 64 is also deposited on the second line such that the second line is covered during activation. In FIG. 3A, active areas are to be formed on the protrusions 28 that alternate along the (first) lines of the single layer base support 42 with the depressions 28, and thus it is desirable to activate the protrusions 28. The protrusions 28 may be activated via plasma ashing or silanization. After activation, the protrusions 28 contain surface groups to attach to the polymeric hydrogel (12A as shown in FIG. 3A or 12B as shown in FIG. 9C). In contrast, the continuous non-patterned regions of the single layer base support 42 that form the non-reactive regions 26, 26′ are coated with the mask material 64.

In the example shown in FIG. 4A, before the active areas (e.g., 10A) are formed over the protrusions 28, the depressions 30 are surrounded by non-patterned regions of the single layer base support 42. In this example, activating the single layer base support 42 involves selectively silanizing the non-patterned regions 62′ (of the layer single layer base support 42) to generate rows and columns of alternating depressions 30 (which have polymeric hydrogel 12A (FIG. 9C) or 12B (FIG. 4A) therein) and activated regions 62′ of the single layer base support 42, wherein the depressions 30 and the activated regions 62′ are circular and have the same diameter. This activation process forms a pattern for the active areas that are to be formed on the protrusions 28.

The other polymeric hydrogel can then be applied, e.g., to the activated protrusions 28 (regions 62′). In FIG. 9C, the other polymeric hydrogel is the second polymeric hydrogel 12B. In one example, the second polymeric hydrogel 12B attaches to the activated portions 62′ on the protrusions 28 and also applies over the mask material 64 and the portion 66′ of the photoresist 66. The second polymeric hydrogel 12B may be applied using any suitable deposition technique.

Removal of the photoresist portion 66′ and the mask material 64 may then be performed.

The photoresist portion 66′ may be lifted off with a positive or negative photoresist remover, such as dimethylsulfoxide (DMSO) using sonication, or acetone, or an NMP (N-methyl-2-pyrrolidone) based stripper. A positive photoresist portion 66′ may also be removed with propylene glycol monomethyl ether acetate. This lift-off process removes i) at least 99% of the insoluble photoresist portion 66′ and ii) the second polymeric hydrogel 12B thereon. This lift-off process exposes the polymeric hydrogel 12A in the depression 30.

The mask material 64 may also be exposed to a lift-off process. Any suitable wet lift-off process may be used, such as soaking, sonication, or spin and dispensing of a lift-off liquid. This lift-off process removes i) the mask material 64 and ii) the second polymeric hydrogel 12B thereon. When this method is used to form the example shown in FIG. 3A, the mask material 64 removal exposes the non-reactive regions 26, 26′.

This method also includes attaching respective primer sets 14A, 14B to the polymeric hydrogels 12A, 12B. In some examples, the primer sets 14A, 14B may be pre-grafted to the respective polymeric hydrogels 12A, 12B. In these examples, additional primer grafting is not performed.

In other examples, the primer sets 14A, 14B are not pre-grafted to the respective polymeric hydrogels 12A, 12B. In these examples, the primer set 14A may be grafted after the polymeric hydrogel 12A is applied (e.g., at FIG. 9B). In these examples, the primer set 14B may be pre-grafted to the second polymeric hydrogel 12B. Alternatively, in these examples, the primer set 14B may not be pre-grafted to the second polymeric hydrogel 12B. Rather, the primer set 14B may be grafted after the second polymeric hydrogel 12B is applied (e.g., at FIG. 9C) and before the lift-off processes are performed. When grafting is performed during the method, grafting may be accomplished using any suitable grafting techniques.

The architecture of the patterned structures 23B, 23D shown in FIG. 3C and FIG. 4C may be prepared by the method shown in FIG. 10A through FIG. 10C. This method generally includes depositing a first polymeric hydrogel 12A on a multi-layer stack including a substrate 22 and a mask material 64 patterned on the substrate 22 to define alternating first areas 68 and second areas 70, the first areas 68 exposing portions of the substrate 22 including surface groups to attach to the first polymeric hydrogel 12A and the second areas 70 being covered by the mask material 64, whereby the first polymeric hydrogel 12A selectively attaches to the portions of the substrate 22 exposed at the first areas 68; lifting off the mask material 64 to expose the second areas 70; activating the second areas 70 with surface groups to attach a second polymeric hydrogel 12B; depositing the second polymeric hydrogel 12B such that it selectively attaches to the second areas 70; grafting a first primer set 14A to the first polymeric hydrogel 12A; and grafting a second primer set 14B to the second polymeric hydrogel 12B, the second primer set 14B being different from the first primer set 14A.

In this example method, the mask material 64 is applied to the substrate 22 (which may be a single layer base support 42 or a multi-layer structure including both the base support 42 and the layer 44 thereon).

The arrangement of the mask material 64 depends upon the desired architecture for the active areas 10A, 10B that are to be formed.

To generate the patterned structure shown in FIG. 3C, the first areas 68 and the second areas 70 are defined along a plurality of first lines (corresponding to the regions 24, 24′, 24″ in FIG. 3C) extending along the substrate 22; and the method further comprises applying a second mask material (not show) along a second line (corresponding to the regions 26, 26′ in FIG. 3C) separating one of the plurality of first lines from an adjacent one of the plurality of first lines, the second line defining a continuous non-patterned region of the substrate 22 that extends a length of each of the plurality of first lines. As such, the mask material 64 is deposited on the areas 70 and not on the areas 68 along the plurality of first lines, and a second mask material is deposited along the second lines.

In this example, the mask material 64 and the second mask material are different materials that can be lifted off under different conditions. As such, the removal of the mask material 64 will not remove the second mask material. Therefore, the second mask material remains in place during the activation of the second areas 70, and can be lifted off in a separate lift-off process.

In this example, with the mask material 64 covering the portions 70 and the second mask material covering the lines (corresponding to the regions 26, 26′ in FIG. 3C), the exposed portions 68 of the substrate 22 (which is layer 44 in FIG. 10A) may be activated via plasma ashing to introduce surface groups that can attach to the first polymeric hydrogel 12A. The first polymeric hydrogel 12A is deposited on the activated exposed portions 68 using any suitable deposition technique. In this example, the first polymeric hydrogel 12A selectively attaches to the active exposed portion(s) 68, and also deposits over the mask material 64 and the second mask material.

To generate the patterned structure shown in FIG. 4C, the first areas 68 and the second areas 70 are formed so that they extend in rows (corresponding to the rows 34, 34′, 34″ in FIG. 4C) and columns (corresponding to the columns 36, 36′, 36″ in FIG. 4C) across the substrate 22. As such, the mask material 64 is deposited on the areas 70 in a circular pattern and not on the areas 68. To form the circular pattern of the areas 68, these areas 68 may be activated. This activation process is selectively performed so that the outer perimeter of the substrate 22 (e.g., region 26 in FIG. 4C) and the regions at the intersection of four first areas 68 and second areas 70 are not activated. The activation of the first areas 68 involves selectively ashing the first areas 68 such that the activated first area 68 is circular and have the same diameter as the second areas 70 (covered by the mask material 64). Selective plasma ashing introduces surface groups to the areas 68 that can attach to the first polymeric hydrogel 12A. In this example, the outer perimeter of the substrate 22 (e.g., region 26 in FIG. 4C) and the regions at the intersection of four first areas 68 and second areas 70 are not activated and thus cannot attach to the first polymeric hydrogel 12A. The first polymeric hydrogel 12A is deposited on the activated exposed portions 68 using any suitable deposition technique. In this example, the first polymeric hydrogel 12A selectively attaches to the active exposed portion(s) 68, and also deposits over the mask material 64.

The deposited polymeric hydrogel 12A is shown in FIG. 10B.

In this example method, the mask material 64 is lifted off to expose the second areas 70. When the second mask material is used along the second line(s) (corresponding to the regions 26, 26′ in FIG. 3C), the second mask material remains in place during the lift off of the mask material 64. Examples of suitable lift off conditions for silicon include basic (pH) conditions, for aluminum include acidic or basic conditions, for gold include an iodine and iodide mixture, for silver include an iodine and iodide mixture, for titanium include H₂O₂), or for copper include an iodine and iodide mixture. Removal of the mask material 64 also removes the first polymeric hydrogel 12A positioned thereon.

The exposed second areas 70 may then be activated. When the second mask material is still in place over the second line(s) (corresponding to the regions 26, 26′ in FIG. 3C), plasma ashing or silanization may be used to activate the second areas 70. When the mask material 64 is removed and thus the second areas 70, the outer perimeter of the substrate 22 (e.g., region 26 in FIG. 4C), and the regions at the intersection of four first areas 68 and second areas 70 are exposed, selective plasma ashing or selective silanization may be used to activate the second areas 70 without activating the outer perimeter of the substrate 22 (e.g., region 26 in FIG. 4C), and the regions at the intersection of four first areas 68 and second areas 70.

The other polymeric hydrogel can then be applied, e.g., to the activated areas 70. In FIG. 10C, the other polymeric hydrogel is the second polymeric hydrogel 12B. The second polymeric hydrogel 12B selectively attaches to the activated portions 70. The second polymeric hydrogel 12B may be applied using any suitable deposition technique, and when the deposition is performed under high ionic strength (e.g., in the presence of 10×PBS, NaCl, KCl, etc.), the second polymeric hydrogel 12B does not deposit on or adhere to the first polymeric hydrogel 12A attached at the regions 68.

When the second mask material is used (e.g., to form the architecture shown in FIG. 3C), the second polymeric hydrogel 12B may also be applied on the second mask material. However, the second mask material may be removed using any suitable wet lift off process for the material, and this will remove both the mask material and the second polymeric hydrogel 12B.

When the method is used to form the architecture in FIG. 4C, the second polymeric hydrogel 12B may be deposited on the outer perimeter of the substrate 22 (e.g., region 26 in FIG. 4C), and on the regions at the intersection of four first areas 68 (with polymeric hydrogel 12A attached thereto) and second areas 70 (with polymeric hydrogel 12B attached thereto). Because these substrate regions have not been activated, the polymeric hydrogel 12B is not covalently attached and can be readily remove via sonication, washing, wiping, etc.

This method also includes attaching respective primer sets 14A, 14B to the polymeric hydrogels 12A, 12B. In some examples, the primer sets 14A, 14B may be pre-grafted to the respective polymeric hydrogels 12A, 12B. In these examples, additional primer grafting is not performed.

In other examples, the primer sets 14A, 14B are not pre-grafted to the respective polymeric hydrogels 12A, 12B. In these examples, the primer set 14A may be grafted after the polymeric hydrogel 12A is applied (e.g., at FIG. 10B). In these examples, the primer set 14B may be pre-grafted to the second polymeric hydrogel 12B. Alternatively, in these examples, the primer set 14B may not be pre-grafted to the second polymeric hydrogel 12B. Rather, the primer set 14B may be grafted after the second polymeric hydrogel 12B is applied (e.g., at FIG. 10C). When grafting is performed during the method, grafting may be accomplished using any suitable grafting techniques.

The architecture of the patterned structure 23E shown in FIG. 5 may be prepared by the methods shown and described in reference to FIG. 10A through FIG. 10C, except that the substrate 22 includes alternating regions (e.g., 38, 38′, 38″ shown in FIG. 5) of a first height and regions (e.g., 40, 40′ shown in FIG. 5) of a second height; and the alternating first areas 68 and second areas 70 (FIG. 10A) extend across each of the regions (e.g., 38, 38′, 38″ shown in FIG. 5) of the first height and the regions of the second height (e.g., 40, 40′ shown in FIG. 5). In one example, if the second mask material is used, it may be applied at the perimeter of the substrate 22 (as there are no other non-reactive regions 26, 26′). In another example, if the second mask material is not used, selective plasma ashing or selective silanization may be used to activate the second areas 70 without activating the outer perimeter of the substrate 22.

The architecture of the patterned structure 23F shown in FIG. 6 may be prepared by the method shown in FIG. 11A through FIG. 11D. This method generally includes depositing a first polymeric hydrogel 12A on a multi-layer stack including a substrate 22 and a first mask material 64 and a different second mask material 64′ patterned on the substrate 22 to define a plurality of first areas 68, each first area 68 exposing a portion of the substrate 22 and being isolated from each other first area 68; a plurality of second areas 70, each second area 70 being covered by the first mask material 64 and being isolated from each other second area 70; and a plurality of third areas 72, each third area 72 being covered by the different second mask material 64′ and being isolated from each other third area 72, whereby the first polymeric hydrogel 12A selectively attaches to the portion of the substrate 22 exposed at each of the plurality of first areas 68; lifting off the first mask material 64 to expose the plurality of second areas 70; activating the plurality of second areas 70 with surface groups to attach a second polymeric hydrogel 12B; depositing the second polymeric hydrogel 12B such that it selectively attaches to the plurality of second areas 70; lifting off the different second mask material 64′ to expose the plurality of third areas 72; activating the plurality of third areas 72 with surface groups to attach a third polymeric hydrogel 12C; depositing the third polymeric hydrogel 12C such that it selectively attaches to the plurality of third areas 72; grafting a first primer set 14A to the first polymeric hydrogel 12A; grafting a second primer set 14B to the second polymeric hydrogel 12B, the second primer set 12B being different from the first primer set 12A; and grafting a third primer set 14C to the third polymeric hydrogel 12C, the third primer set 14C being different from the first primer set 14A and the second primer set 14B.

In this example method, the mask materials 64, 64′ are applied to the substrate 22 (which may be a single layer base support 42 or a multi-layer structure including both the base support 42 and the layer 44 thereon).

The arrangement of the mask materials 64, 64′ depends upon the desired architecture for the active areas 10A, 10B, 10C that are to be formed.

In this example, the shape of the mask materials 64, 64′ in the x-y plane of the substrate 22 may be a circle. In this example, the mask materials 64, 64′ are different materials that can be lifted off under different conditions. As such, the removal of the mask material 64 will not remove the mask material 64′. Therefore, the mask material 64′ remains in place during the activation of the second areas 70, and can be lifted off in a separate lift-off process.

In this example, with the mask material 64 covering the portions 70 and the mask material 64′ covering the portions 72, the exposed portions 68 of the substrate 22 (which is layer 44 in FIG. 11A) may be activated via plasma ashing to introduce surface groups that can attach to the first polymeric hydrogel 12A. In this example, the outer perimeter of the substrate 22 (e.g., region 26 in FIG. 6) and the regions at the intersection of three first areas 68, 70, 72 are not activated and thus cannot attach to the first polymeric hydrogel 12A. The first polymeric hydrogel 12A is deposited on the activated exposed portions 68 using any suitable deposition technique. In this example, the first polymeric hydrogel 12A selectively attaches to the active exposed portion(s) 68, and also deposits over the mask materials 64, 64′ as shown in FIG. 11B.

In this example method, the mask material 64 is lifted off to expose the second areas 70. Examples of suitable lift off conditions for silicon include basic (pH) conditions, for aluminum include acidic or basic conditions, for gold include an iodine and iodide mixture, for silver include an iodine and iodide mixture, for titanium include H₂O₂), or for copper include an iodine and iodide mixture. Removal of the mask material 64 also removes the first polymeric hydrogel 12A positioned thereon.

The exposed second areas 70 may then be activated, as shown in FIG. 11C. Selective plasma ashing or selective silanization may be used to activate the second areas 70 without activating the outer perimeter of the substrate 22 (e.g., region 26 in FIG. 6), and the regions at the intersection of a first area 68, a second area 70, and a third area 72.

The second polymeric hydrogel 12B can then be applied, e.g., to the activated areas 70. This is shown in FIG. 11C. The second polymeric hydrogel 12B selectively attaches to the activated portions 70. The second polymeric hydrogel 12B may be applied using any suitable deposition technique, and when the deposition is performed under high ionic strength (e.g., in the presence of 10×PBS, NaCl, KCl, etc.), the second polymeric hydrogel 12B does not deposit on or adhere to the first polymeric hydrogel 12A attached at the regions 68.

The second polymeric hydrogel 12B may be deposited on the outer perimeter of the substrate 22 (e.g., region 26 in FIG. 6), and on the regions at the intersection of a first area 68 (with polymeric hydrogel 12A attached thereto), a second area 70 (with polymeric hydrogel 12B attached thereto), and a third area 72. Because these substrate regions have not been activated, the polymeric hydrogel 12B is not covalently attached and can be readily remove via sonication, washing, wiping, etc.

In this example method, the mask material 64′ is lifted off to expose the third areas 72. Any suitable lift off conditions may be used, depending upon the mask material 64′. Removal of the mask material 64′ also removes the second polymeric hydrogel 12B positioned thereon.

The exposed third areas 72 may then be activated, as shown in FIG. 11D. Selective plasma ashing or selective silanization may be used to activate the third areas 72 without activating the outer perimeter of the substrate 22 (e.g., region 26 in FIG. 6), and the regions at the intersection of a first area 68, a second area 70, and a third area 72.

The third polymeric hydrogel 12C can then be applied, e.g., to the activated areas 72. This is shown in FIG. 11D. The third polymeric hydrogel 12C selectively attaches to the activated portions 72. The third polymeric hydrogel 12C may be applied using any suitable deposition technique, and when the deposition is performed under high ionic strength (e.g., in the presence of 10×PBS, NaCl, KCl, etc.), the third polymeric hydrogel 12C does not deposit on or adhere to the first polymeric hydrogel 12A attached at the regions 68 or the second polymeric hydrogel at the portions 72.

The third polymeric hydrogel 12C may be deposited on the outer perimeter of the substrate 22 (e.g., region 26 in FIG. 6), and on the regions at the intersection of a first area 68 (with polymeric hydrogel 12A attached thereto), a second area 70 (with polymeric hydrogel 12B attached thereto), and a third area 72. Because these substrate regions have not been activated, the polymeric hydrogel 12C is not covalently attached and can be readily remove via sonication, washing, wiping, etc.

This method also includes attaching respective primer sets 14A, 14B, 14C to the polymeric hydrogels 12A, 12B, 12C. In some examples, the primer sets 14A, 14B, 14C may be pre-grafted to the respective polymeric hydrogels 12A, 12B, 12C. In these examples, additional primer grafting is not performed.

In other examples, the primer sets 14A, 14B, 14C are not pre-grafted to the respective polymeric hydrogels 12A, 12B, 12C. In these examples, the primer set 14A may be grafted after the polymeric hydrogel 12A is applied (e.g., at FIG. 11B). In these examples, the primer set 14B may be pre-grafted to the second polymeric hydrogel 12B and the primer set 14C may be pre-grafted to the third polymeric hydrogel 12C. Alternatively, in these examples, the primer set 14B may not be pre-grafted to the second polymeric hydrogel 12B and the primer set 14C may not be pre-grafted to the third polymeric hydrogel 12C. Rather, the primer set 14B may be grafted after the second polymeric hydrogel 12B is applied (e.g., at FIG. 11C), and the primer set 14C may be grafted after the third polymeric hydrogel 12C is applied (e.g., at FIG. 11D). When grafting is performed during the method, grafting may be accomplished using any suitable grafting techniques.

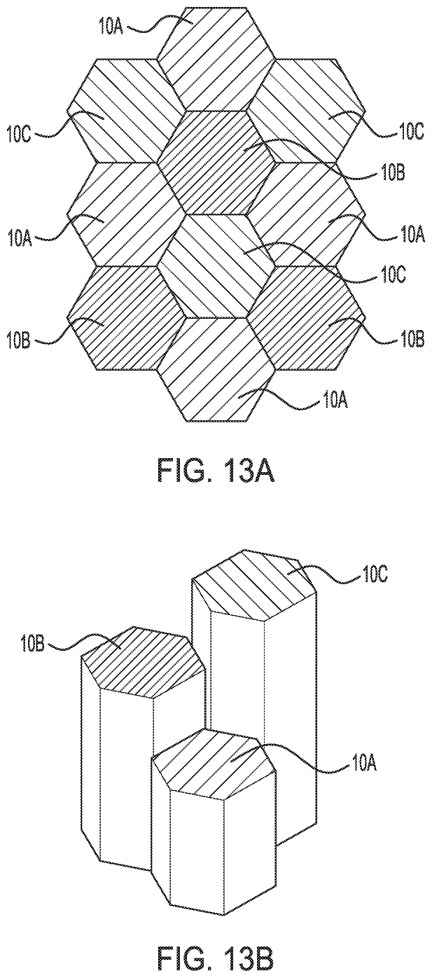

The architecture shown in FIG. 6 may also be prepared using a multi-depth substrate having depressions 30 of different depths. The active areas 10A may be formed in depression 30 with a first depth, the active areas 10B may be formed in depressions 30 with a second depth (e.g., first depth−(minus) 150 nm), and the active areas 10C may be formed in depressions 30 with a third depth (e.g., second depth−150 nm) or on the substrate surface. In one example, these depressions are hexagonal shaped, and thus the active areas 10A, 10B, 10C are also hexagonal shaped (see, e.g., FIG. 13A depicting a top view and FIG. 13B depicting a perspective view of a portion of the substrate). This architecture may be formed by the method shown in FIG. 12A through FIG. 12L. The method utilizes a resin (e.g., layer 44, 44′ of a multi-layered structure) whose ultraviolet light absorbance can be varied with thickness. In these examples, the thinner portions may be UV transparent while the thicker portions may be UV absorbent. This allows the resin to be used as a mask for backside patterning of photoresist materials.

In the method of FIG. 12A through FIG. 12L, the UV absorbance of the resin layer 44′ can be altered by adjusting its thickness. Any of the previously listed resins may be used so long as thicker portions absorb the UV light and thinner portions transmit a desirable amount of UV light for patterning when the resin is exposed to a predetermined UV light dosage. In one example, a polyhedral oligomeric silsesquioxane based resin having thicker portions of about 500 nm and thinner portions of about 150 nm will respectively and effectively absorb and transmit UV light when exposed to a dosage ranging from about 30 mJ/cm² to about 60 mJ/cm². Other thicknesses may be used, and the UV dosage may be adjusted accordingly to achieve the desired absorption in thicker areas and transmittance in thinner areas.

While not shown in FIG. 12A through FIG. 12L, the resin layer 44′ may be supported by any example of the base support 42 set forth herein that is capable of transmitting the UV light. In this example, the thick and thin portions of the resin layer 44′ are adjusted to achieve the desired absorption and transmittance.

The correlation between UV dose, UV absorption constant, and resin layer thickness can be expressed as:

D₀=D×exp(−kd)

where D₀ is the required UV dose to pattern the resin layer, D is the actual UV dose which has to be applied to the resin layer, k is the absorption constant, and d is the thickness of thinner portion of the resin layer. Thus, the actual UV dose (D) can be expressed as:

D₀=D₀/exp(−kd)

In one example, the resin layer 44′ is the negative photoresist NR9-1000P (from Futurrex), D₀=19 mJ/cm² at 0.9 μm of thickness, the UV absorption constant (k) of the photoresist is 3×10⁴ cm⁻¹, the thickness of the thinner portion of photoresist is 150 nm, and D is about 30 mJ/cm².

In FIG. 12A through FIG. 12C, the method shown generally includes: depositing a first polymeric hydrogel 12A over the resin layer 44′ including a plurality of multi-depth depressions 30′ separated by interstitial regions 74, each multi-depth depression 30′ including a deep portion 76 and a shallow portion 78 adjacent to the deep portion 76 (FIG. 12B); depositing and curing a photoresist 66 over the first functionalized layer 24; and timed dry etching to remove the photoresist 66 and the first polymeric hydrogel 12A from the shallow portions 78 and the interstitial regions 74 (FIG. 12C).

The multi-depth depression 30′ shown in FIG. 3A may be etched, imprinted, or defined in the resin layer 44′ using any suitable technique. In one example, nanoimprint lithography is used. In this example, a working stamp is pressed into the resin layer 44′ while the material is soft, which creates an imprint (negative replica) of the working stamp features in the resin layer 44′. The resin layer 44′ may then be cured with the working stamp in place. Curing may be accomplished by exposure to actinic radiation, such as visible light radiation or ultraviolet (UV) radiation, when a radiation-curable resin material is used; or by exposure to heat when a thermal-curable resin material is used. Curing may promote polymerization and/or cross-linking. As an example, curing may include multiple stages, including a softbake (e.g., to drive off any liquid carrier that may be used to deposit the resin) and a hardbake. The softbake may take place at a lower temperature, ranging from about 50° C. to about 150° C., for greater than 0 seconds to about 3 minutes. The duration of the hardbake may last from about 5 seconds to about 10 minutes at a temperature ranging from about 100° C. to about 300° C. Examples of devices that can be used for softbaking and/or hardbaking include a hot plate, oven, etc.

After curing, the working stamp is released. This creates topographic features in the resin layer 44′. In this example, the topographic features of the multi-depth depression 30′ include the shallow portion 78 and the deep portion 76.

While two multi-depth depressions 30′ are shown in FIG. 12A, it is to be understood that the method may be performed to generate an array of multi-depth depressions 30′ including respective deep portions 76 and shallow portions 78, separated by interstitial regions 74, across the surface of the resin layer 44′.

As shown in FIG. 12B, the first polymeric layer 12A is deposited over the resin layer 44′. The first polymeric layer 12A may be any of the gel materials described herein and may be applied using any suitable deposition technique. A curing process may be performed after deposition. The first polymeric layer 12A covalently attaches to the resin layer 44′. Covalent linking is helpful for maintaining the primer set 14A in the desired regions throughout the lifetime of the flow cell 20 during a variety of uses.

To arrive at the structure in FIG. 12C, a positive or negative photoresist 66 is applied over the first polymeric hydrogel 12A. In this example, the entire photoresist 66 may be developed to form an insoluble portion, so that it can be exposed to timed dry etching. The timed dry etching process is used to remove portions of the photoresist 66 and the first polymeric hydrogel 12A from the interstitial regions 74 and from the shallow portions 78 of the multi-depth depressions 30′. As shown in FIG. 12C, timed dry etching is stopped so that a portion of the polymeric hydrogel 12A and a portion 66′ of the photoresist 66 remain in the deep portions 76 of the depressions 30′. In one example, the timed dry etch may involve a reactive ion etch (e.g., with CF₄) where the photoresist 66 is etched at a rate of about 17 nm/min. In another example, the timed dry etch may involve a 100% O₂ plasma etch where the photoresist 66 is etched at a rate of about 98 nm/min.

As mentioned, during the etching of the photoresist 66, the polymeric hydrogel 12A over the interstitial regions 74 and in the shallow portions 78 may also be removed. A combustion reaction may be taking place, where the polymeric hydrogel 12A is converted to carbon dioxide and water and is evacuated from the etching chamber.

The portion 66′ of the photoresist 66 may then be lifted off. The photoresist portion 66′ may be lifted off with a positive or negative photoresist remover, such as dimethylsulfoxide (DMSO) using sonication, or acetone, or an NMP (N-methyl-2-pyrrolidone) based stripper. A positive photoresist portion 66′ may also be removed with propylene glycol monomethyl ether acetate. This lift-off process removes i) at least 99% of the insoluble photoresist portion 66′, leaving the polymeric hydrogel 12A in the deep portions 76 of the multi-depth depressions 30′ (as shown in FIG. 12D).

A negative photoresist 80 is then applied over the resin layer 44′, including over the interstitial regions 74, in the shallow portions 78, and over the first polymeric hydrogel 12A in the deep portions 76. Ultraviolet light is then directed through the backside of the resin layer 44′ to pattern the negative photoresist 80 and generate an insoluble photoresist 80′ and a soluble photoresist 80″. While not shown, any base support that is used is able to transmit the UV light used for the backside exposure.

The first thickness t₁ of the resin layer 44′ is selected to allow the dose of UV light to transmit through the resin layer 44′ and the second and third thicknesses t₂, t₃ are selected to block the dose of UV light from transmitting through the resin layer 44′. As such, the portion of the negative photoresist 80 overlying the thickness t₁ becomes insoluble (80′) due to the exposure to the UV light, and the portions of the negative photoresist 80 overlying the thicknesses t₂, t₃ become soluble (80″) due to the lack of exposure to the UV light. In other words, when exposed to the ultraviolet light dosage, the insoluble negative photoresist 80′ forms in the deep portions 76 and the soluble negative photoresist 80″ forms in the shallow portions 78 and over the interstitial regions 74.

The soluble negative photoresist 80″ is then removed using any suitable developer described herein for negative photoresists. The removal of the soluble negative photoresist 80″ exposes the resin layer 44′ at the shallow portions 78 and the interstitial regions 74. This is shown in FIG. 12F.

As shown in FIG. 12F, the second polymeric layer 12B is deposited over the resin layer 44′. The second polymeric layer 12B may be any of the gel materials described herein and may be applied using any suitable deposition technique. A curing process may be performed after deposition. The second polymeric layer 12B covalently attaches to the resin layer 44′.

FIG. 12G depicts the removal of the insoluble negative photoresist 80′. The insoluble negative photoresist 80′ may be removed via a lift-off process. The lift-off process may be any suitable lift-off process described herein, and may involve a suitable remover for the type of negative photoresist 80 used. As shown in FIG. 12G, the removal process removes i) at least 99% of the insoluble photoresist 80′ and ii) the second polymeric hydrogel 12B applied thereon. This removal process leaves the second polymeric hydrogel 12B that is positioned in the shallow portion 78 and over the interstitial regions 74 intact, and also leaves the first polymeric hydrogel 12A intact. These portions of the polymeric hydrogels 12A, 12B remain intact, in part because they are covalently attached to the resin layer 44′.

To arrive at the structure in FIG. 12H, another positive or negative photoresist 66 is applied over the resin layer 44′, including over the first and second polymeric hydrogels 12A, 12B. In this example, the entire photoresist 66 may be developed to form an insoluble portion, so that it can be exposed to timed dry etching. The timed dry etching process is used to remove portions of the photoresist 66 and the second polymeric hydrogel 12B from the interstitial regions 74. As shown in FIG. 12H, timed dry etching is stopped so that a portion of the polymeric hydrogel 12B remains in the shallow portions 78, the portion of the polymeric hydrogel 12A remains in the deep portions 76, and a portion 66′ of the photoresist 66 remains over both of the polymeric hydrogels 12A, 12B. In one example, the timed dry etch may involve a reactive ion etch (e.g., with CF₄) where the photoresist 66 is etched at a rate of about 17 nm/min. In another example, the timed dry etch may involve a 100% O₂ plasma etch where the photoresist 66 is etched at a rate of about 98 nm/min.

As mentioned, during the etching of the photoresist 66, the second polymeric hydrogel 12B over the interstitial regions 74 may also be removed. A combustion reaction may be taking place, where the polymeric hydrogel 12B is converted to carbon dioxide and water and is evacuated from the etching chamber.

The portion 66′ of the photoresist 66 may then be lifted off. The photoresist portion 66′ may be lifted off with a positive or negative photoresist remover, such as dimethylsulfoxide (DMSO) using sonication, or acetone, or an NMP (N-methyl-2-pyrrolidone) based stripper. A positive photoresist portion 66′ may also be removed with propylene glycol monomethyl ether acetate. This lift-off process removes i) at least 99% of the insoluble photoresist portion 66′, leaving the polymeric hydrogel 12A in the deep portions 76 and polymeric hydrogel 12B in the shallow portions 78 of the multi-depth depressions 30′ (as shown in FIG. 12H).

Another negative photoresist 80 is then applied over the resin layer 44′, including over the interstitial regions 74, over the second polymeric hydrogel 12B in the shallow portions 78, and over the first polymeric hydrogel 12A in the deep portions 76. This is shown in FIG. 12J. Ultraviolet light is then directed through the backside of the resin layer 44′ to pattern the negative photoresist 80 and generate an insoluble photoresist 80′ and a soluble photoresist 80″.

It is to be understood that the UV light dosage used during this process is more intense than the UV light dosage used to pattern the negative photoresist 80 during the process described in reference to FIG. 12E. Thus, in FIG. 12J, both the first thickness t₁ and the second thickness t₂ of the resin layer 44′ allow the higher dose of UV light to transmit through the resin layer 44′ and the third thicknesses t₃ blocks the higher dose of UV light from transmitting through the resin layer 44′. As such, the portions of the negative photoresist 80 overlying the thicknesses t₁, t₂ become insoluble (80′) due to the exposure to the UV light, and the portion of the negative photoresist 80 overlying the thickness t₃ become soluble (80″) due to the lack of exposure to the UV light. In other words, when exposed to the higher ultraviolet light dosage, the insoluble negative photoresist 80′ forms in the deep portions 76 and in the shallow portions 78 and the soluble negative photoresist 80″ forms over the interstitial regions 74 (see FIG. 12J).

The soluble negative photoresist 80″ is then removed using any suitable developer described herein for negative photoresists. The removal of the soluble negative photoresist 80″ exposes the resin layer 44′ at the interstitial regions 74. This is shown in FIG. 12K.

As shown in FIG. 12K, the third polymeric layer 12C is deposited over the resin layer 44′. The third polymeric layer 12C may be any of the gel materials described herein and may be applied using any suitable deposition technique. A curing process may be performed after deposition. The third polymeric layer 12C covalently attaches to the resin layer 44′ (e.g., at the interstitial regions 74).

FIG. 12L depicts the removal of the insoluble negative photoresist 80′. The insoluble negative photoresist 80′ may be removed via a lift-off process. The lift-off process may be any suitable lift-off process described herein, and may involve a suitable remover for the type of negative photoresist 80 used. As shown in FIG. 12L, the removal process removes i) at least 99% of the insoluble photoresist 80′ and ii) the third polymeric hydrogel 12C applied thereon. This removal process leaves the second polymeric hydrogel 12B that is positioned in the shallow portion 78 intact, and also leaves the first polymeric hydrogel 12A that is positioned in the deep portion 76 intact.

This method also includes attaching respective primer sets 14A, 14B, 14C to the polymeric hydrogels 12A, 12B, 12C. In some examples, the primer sets 14A, 14B, 14C may be pre-grafted to the respective polymeric hydrogels 12A, 12B, 12C. In these examples, additional primer grafting is not performed.

In other examples, the primer sets 14A, 14B, 14C are not pre-grafted to the respective polymeric hydrogels 12A, 12B, 12C. In these examples, the primer set 14A may be grafted after the polymeric hydrogel 12A is applied (e.g., at FIG. 12B). In these examples, the primer set 14B may be pre-grafted to the second polymeric hydrogel 12B and the primer set 14C may be pre-grafted to the third polymeric hydrogel 12C. Alternatively, in these examples, the primer set 14B may not be pre-grafted to the second polymeric hydrogel 12B and the primer set 14C may not be pre-grafted to the third polymeric hydrogel 12C. Rather, the primer set 14B may be grafted after the second polymeric hydrogel 12B is applied (e.g., at FIG. 12F), and the primer set 14C may be grafted after the third polymeric hydrogel 12C is applied (e.g., at FIG. 12K). When grafting is performed during the method, grafting may be accomplished using any suitable grafting techniques.

The architecture shown in FIG. 7A through FIG. 7D may be prepared by incorporating the capture primers 48 at desired areas on the substrate 22 (e.g., using a binding pair, such as streptavidin and biotin, or other suitable attachment mechanism), and depositing the polymeric hydrogel 12 such that is surrounds the capture primers 48. The primer set 14 may be pre-grafted or grafted after the polymeric hydrogel 12 is deposited.

Methods for Using the Flow Cells

Some examples of the flow cell 20 disclosed herein including the primer sets 14A, 14B, and in some instances 14C attached to the polymers 12A, 12B, 12C may be used in a sequential paired-end read sequencing method. Different library fragments introduced into the flow cell 20 are able to seed and amplify at each of the active areas 10A, 10B, 10C. Because of the different primers sets 14A, 14B, 14C, the amplification of any given library fragment across a respective active area 10A, 10B, 10C is not able to continue on an adjacent, but different active area 10B, 10C, 10A. In this method, the respective forward strands that are generated on a particular active area 10A, 10B, 10C are sequenced and removed, and then the respective reverse strands are sequenced and removed.

When the flow cell 20 includes the primer subset 13A, 15A, or 13B, 15B, or 13C, 15C, or 13D, 15D attached to the polymeric hydrogel regions 12A1, 12A2 of an active area 10A, 10B or 10A (instead of one of the primer sets 14A, 14B, 4C), the subset may be used in a simultaneously paired-end read sequencing method. As described herein, the primer subsets 13A, 15A, or 13B, 15B, or 13C, 15C, or 13D, 15D are controlled so that the cleaving (linearization) chemistry is orthogonal at the different polymeric hydrogel regions 12A1, 12A2. This enables a cluster of forward strands to be generated in one region 12A1 of the active area 10A and a cluster of reverse strands to be generated in another region 12A2 of the active area 10A. In an example, the regions 12A1, 12A2 are directly adjacent to one another, and are adjacent to an orthogonal active area 10B, 10C. This enables simultaneous paired-end reads to be obtained on the active area 10A.

### ADDITIONAL NOTES

It should also be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. Moreover, it is to be understood that any features of any of the examples disclosed herein may be combined together in any desirable manner and/or configuration.

It should also be appreciated that terminology explicitly employed herein that also may appear in any disclosure incorporated by reference should be accorded a meaning most consistent with the particular concepts disclosed herein.

Reference throughout the specification to “one example”, “another example”, “an example”, and so forth, means that a particular element (e.g., feature, structure, and/or characteristic) described in connection with the example is included in at least one example described herein, and may or may not be present in other examples. In addition, it is to be understood that the described elements for any example may be combined in any suitable manner in the various examples unless the context clearly dictates otherwise.

It is to be understood that the ranges provided herein include the stated range and any value or sub-range within the stated range, as if such values or sub-ranges were explicitly recited. For example, a range of about 0.35 μm (350 nm) to at least 1.8 μm (1800 nm), should be interpreted to include not only the explicitly recited limits of about 0.35 μm (350 nm) to at least 1.8 μm (1800 nm), but also to include individual values, such as about 0.708 μm (708 nm), about 0.9 μm (900 nm), etc., and sub-ranges, such as from about 0.425 μm (425 nm) to about 0.825 μm (825 nm), from about 0.550 μm (550 nm) to about 0.940 μm (940 nm), etc. Furthermore, when “about” and/or “substantially” are/is utilized to describe a value, they are meant to encompass minor variations (up to +/−10%) from the stated value.

While several examples have been described in detail, it is to be understood that the disclosed examples may be modified. Therefore, the foregoing description is to be considered non-limiting.

1. A flow cell, comprising:
a substrate;
a plurality of reactive regions extending along the substrate; and
a non-reactive region separating one of the plurality of reactive regions from an adjacent one of the plurality of reactive regions;
wherein:
each of the plurality of reactive regions includes alternating first and second areas positioned along the reactive region;
each of the first areas includes a first primer set and each of the second areas includes a second primer set that is different than the first primer set; and
i) adjacent first and second areas directly abut each other or ii) the first areas are positioned on protrusions and the second areas are positioned in depressions adjacent to the protrusions.

2. The flow cell as defined in claim 1, wherein:
the first primer set includes P5 and P7 primers; and
the second primer set includes any combination of PA, PB, PC and/or PD primers.

3. The flow cell as defined in claim 1, wherein:
the first primer set includes unblocked P5 and P7 primers; and
the second primer set includes 3′ blocked P5 and P7 primers.

4. The flow cell as defined in claim 1, wherein the non-reactive region is an exposed portion of the substrate.

5. The flow cell as defined in claim 1, wherein each of the first and second areas includes a polymeric hydrogel to which the respective first and second primer sets are attached.

6. The flow cell as defined in claim 1, wherein:
the first area includes a first polymeric hydrogel to which the first primer set is attached;
the second area includes a second polymeric hydrogel to which the second primer set is attached; and
the first polymeric hydrogel and the second polymeric hydrogel include orthogonal functional groups to respectively attach the first primer set and the second primer set.

7. The flow cell as defined in claim 1, wherein:
the first areas are positioned on the protrusions and the second areas are positioned in the depressions adjacent to the protrusions; and
a sidewall of each of the depressions defines a respective interstitial region.

8. A flow cell, comprising:
a substrate; and
rows and columns of alternating first and second areas;
wherein:
each of the first areas includes a first primer set and each of the second areas includes a second primer set that is different than the first primer set; and
i) adjacent first and second areas directly abut each other or ii) the first areas are positioned on protrusions and the second areas are positioned in depressions adjacent to the protrusions.

9. The flow cell as defined in claim 8, wherein a shape of each of the first and second areas is a circle or a diamond.

10. The flow cell as defined in claim 8, wherein:
the first primer set includes P5 and P7 primers; and
the second primer set includes any combination of PA, PB, PC and/or PD primers.

11. The flow cell as defined in claim 8, wherein:
the first primer set includes unblocked P5 and P7 primers; and
the second primer set includes 3′ blocked P5 and P7 primers.

12. The flow cell as defined in claim 8, wherein each of the first and second areas includes a polymeric hydrogel to which the respective first and second primer sets are attached.

13. The flow cell as defined in claim 8, wherein:
the first area includes a first polymeric hydrogel to which the first primer set is attached;
the second area includes a second polymeric hydrogel to which the second primer set is attached; and
the first polymeric hydrogel and the second polymeric hydrogel include orthogonal functional groups to respectively attach the first primer set and the second primer set.

14. A flow cell, comprising:
a substrate having alternating regions of a first height and regions of a second height; and
alternating first and second areas extending along the regions of the first height and extending along the regions of the second height;
wherein each of the first areas includes a first primer set, and each of the second areas includes a second primer set that is different than the first primer set.

15. The flow cell as defined in claim 14, wherein a difference between the first height and the second height is at least 150 nm.

16. The flow cell as defined in claim 14, wherein:
the first primer set includes P5 and P7 primers; and
the second primer set includes any combination of PA, PB, PC and/or PD primers.

17. The flow cell as defined in claim 14, wherein:
the first primer set includes unblocked P5 and P7 primers; and
the second primer set includes 3′ blocked P5 and P7 primers.

18. The flow cell as defined in claim 14, further comprising a non-reactive region located at at least a portion of the perimeter of the substrate, wherein the non-reactive region is an exposed portion of the substrate.

19. The flow cell as defined in claim 14, wherein each of the first and second areas includes a polymeric hydrogel to which the respective first and second primer sets are attached.

20. The flow cell as defined in claim 14, wherein:
the first area includes a first polymeric hydrogel to which the first primer set is attached;
the second area includes a second polymeric hydrogel to which the second primer set is attached; and
the first polymeric hydrogel and the second polymeric hydrogel include orthogonal functional groups to respectively attach the first primer set and the second primer set.

44. (canceled)


SEQUENCE LISTING (14 sequences)

SEQ ID NO: 1 - DNA, 29 bp, Artificial Sequence
  Synthesized
aatgatacggcgaccaccgagauctacac

SEQ ID NO: 2 - DNA, 24 bp, Artificial Sequence
  Synthesized
  misc_feature (22)..(22): 8-oxoguanine or uracil
caagcagaagacggcatacganat

SEQ ID NO: 3 - DNA, 24 bp, Artificial Sequence
  Synthesized
  misc_feature (20)..(20): 8-oxoguanine or uracil
caagcagaagacggcatacnagat

SEQ ID NO: 4 - DNA, 29 bp, Artificial Sequence
  Synthesized
  misc_feature (23)..(23): allyl-T
aatgatacggcgaccaccgaganctacac

SEQ ID NO: 5 - DNA, 35 bp, Artificial Sequence
  Synthesized
gctggcacgtccgaacgcttcgttaatccgttgag

SEQ ID NO: 6 - DNA, 35 bp, Artificial Sequence
  Synthesized
ctcaacggattaacgaagcgttcggacgtgccagc

SEQ ID NO: 7 - DNA, 35 bp, Artificial Sequence
  Synthesized
cgtcgtctgccatggcgcttcggtggatatgaact

SEQ ID NO: 8 - DNA, 35 bp, Artificial Sequence
  Synthesized
agttcatatccaccgaagcgccatggcagacgacg

SEQ ID NO: 9 - DNA, 35 bp, Artificial Sequence
  Synthesized
acggccgctaatatcaacgcgtcgaatccgcaact

SEQ ID NO: 10 - DNA, 35 bp, Artificial Sequence
  Synthesized
agttgcggattcgacgcgttgatattagcggccgt

SEQ ID NO: 11 - DNA, 34 bp, Artificial Sequence
  Synthesized
gccgcgttacgttagccggactattcgatgcagc

SEQ ID NO: 12 - DNA, 34 bp, Artificial Sequence
  Synthesized
gctgcatcgaatagtccggctaacgtaacgcggc

SEQ ID NO: 13 - DNA, 24 bp, Artificial Sequence
  Synthesized
aggaggaggaggaggaggaggagg

SEQ ID NO: 14 - DNA, 24 bp, Artificial Sequence
  Synthesized
cctcctcctcctcctcctcctcct